(19) United States
(12) Patent Application Publication
(10) Pub. No.: US 2018/0309057 A1
(43) Pub. Date: Oct. 25, 2018

(54) ORGANIC ELECTROLUMINESCENCE DEVICE

(71) Applicant: IDEMITSU KOSAN CO., LTD., Tokyo (JP)

(72) Inventor: Takeshi IKEDA, Sodegaura-shi, Chiba (JP)

(73) Assignee: IDEMITSU KOSAN CO., LTD., Tokyo (JP)

(21) Appl. No.: 15/768,816

(22) PCT Filed: Oct. 20, 2016
(86) PCT No.: PCT/JP2016/081152

§ 371 Date: Apr. 16, 2018

(30) Foreign Application Priority Data
Oct. 20, 2015 (JP) 2015-206432

(51) Int. Cl. H01L 51/00; H01L 51/50

(52) U.S. Cl. CPC H01L 51/006; H01L 51/5056; H01L 51/5072; H01L 51/5092; H01L 51/0085; H01L 51/0074; H01L 51/0088; H01L 51/5036; H01L 51/5024; H01L 51/0055; H01L 51/0073; H01L 51/0087

(57) ABSTRACT

An organic electroluminescence device comprising an anode, a hole-injecting layer, a hole-transporting layer, an emitting layer and a cathode in this order, wherein the hole-transporting layer comprises a compound represented by the following formula (1), and the hole-injecting layer comprises a compound represented by the following formula (100). Wherein in the formula (100), X¹ and X² are independently the following formula (a) or (b).

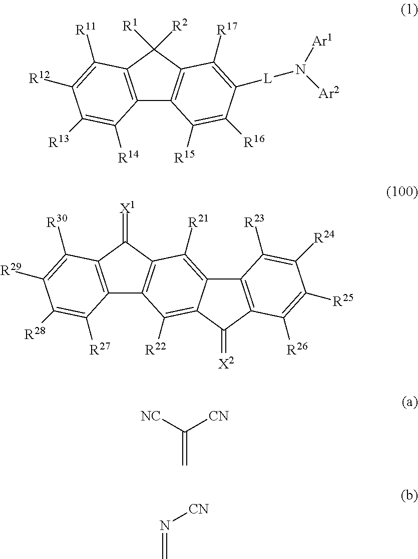

### TECHNICAL FIELD

The invention relates to an organic electroluminescence device and a display apparatus or a light-emitting apparatus using the same.

### BACKGROUND ART

An organic electroluminescence (EL) device is a self-emitting device utilizing the principle that a fluorescent substance emits light by the re-combination energy of holes injected from an anode and electrons injected from a cathode when an electric field is applied.

An organic EL device has a stacked layer structure that further includes, between an anode and a cathode, organic layers such as a hole-injecting layer, a hole-transporting layer, an electron-injecting layer and an electron-transporting layer in addition to an emitting layer. In such device, in order to increase re-combination efficiency of holes and electrons injected, contrivances are made in materials used in each layer or device structure.

For example, as the hole-transporting material and the hole-injecting material, aromatic amine derivatives are known (for example, Patent Document 1). Further, as the hole-injecting material, indenofluorenedione derivatives are known (for example, Patent Document 2).

### RELATED ART DOCUMENTS

### Patent Documents

### Patent Document 1: WO2008/062636

### Patent Document 2: WO2009/011327

### SUMMARY OF THE INVENTION

An object of the invention is to provide an organic EL device that can be driven at a low voltage and has a high luminous efficiency.

According to one aspect of the invention, the following organic EL device is provided.

An organic electroluminescence device comprising an anode, a hole-injecting layer, a hole-transporting layer, an emitting layer and a cathode in this order,

wherein

the hole-transporting layer comprises a compound represented by the following formula (1), and

the hole-injecting layer comprises a compound represented by the following formula (100):

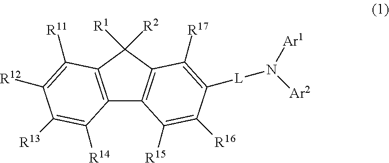

wherein in the formula (1), R¹ and R² are independently an alkyl group including 1 to 4 carbon atoms;

R¹¹ to R¹⁷ are independently a hydrogen atom or a substituent, and adjacent ones of R¹¹ to R¹⁴ may be bonded with each other to form a ring;

Ar¹ and Ar² are independently a substituted or unsubstituted aryl group including 6 to 25 carbon atoms that form a ring (hereinafter referred to as “ring carbon atoms”) or a substituted or unsubstituted heteroaryl group including 6 to 24 atoms that form a ring (hereinafter referred to as “ring atoms”); and

L is a single bond, a substituted or unsubstituted arylene group or a substituted or unsubstituted heteroarylene group;

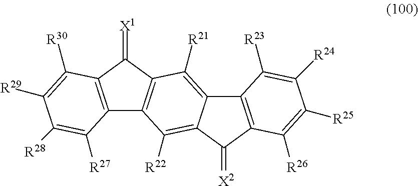

wherein in the formula, X¹ and X² are independently represented by the following formula (a) or (b); and

R²¹ to R³⁰ are independently a hydrogen atom, a substituted or unsubstituted alkyl group including 1 to 20 carbon atoms, a substituted or unsubstituted cycloalkyl group including 3 to 20 carbon atoms, a substituted or unsubstituted alkenyl group including 1 to 20 carbon atoms, a substituted or unsubstituted aryl group including 6 to 50 ring carbon atoms, a substituted or unsubstituted heterocyclic group including 3 to 50 ring atoms, a halogen atom, a substituted or unsubstituted fluoroalkyl group including 1 to 20 carbon atoms, a substituted or unsubstituted alkoxy group including 1 to 20 carbon atoms, a substituted or unsubstituted fluoroalkoxy group including 1 to 20 carbon atoms, a substituted or unsubstituted aryloxy group including 6 to 50 ring carbon atoms, a substituted or unsubstituted aralkyloxy group including 7 to 70 carbon atoms, a substituted or unsubstituted amino group or a cyano group.

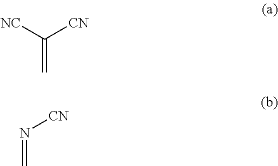

According to another aspect of the invention, a display apparatus or a light-emitting apparatus provided with the above-mentioned organic electroluminescence device is provided.

According to the invention, it is possible to provide an organic EL device that can be driven at a low voltage and has a high luminous efficiency.

### BRIEF DESCRIPTION OF THE DRAWINGS

FIG. 1 is a schematic cross-sectional view of the organic EL device according to one embodiment of the invention.

### MODE FOR CARRYING OUT THE INVENTION

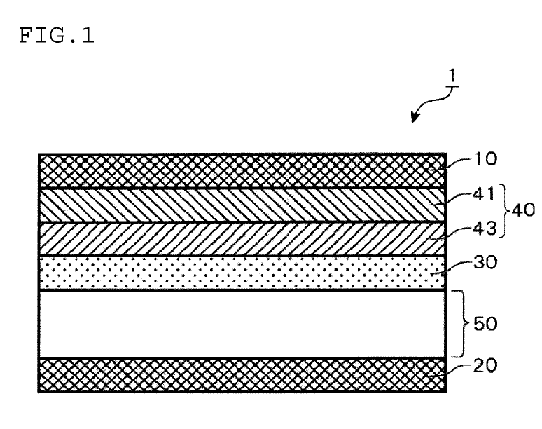

FIG. 1 is a schematic cross-sectional view of the organic EL device according to one embodiment of the invention.

As shown in FIG. 1, an organic EL device 1 of the invention has an anode 10, a hole-injecting layer 41, a hole-transporting layer 43, an emitting layer 30 and a cathode 20 in this order. The hole-injecting layer 41 and the hole-transporting layer 43 form a hole-transporting zone 40, and in the hole-transporting zone 40, other hole-injecting layers and other hole-transporting layers may be provided. It is preferred that an electron-transporting zone 50 be formed between the cathode 20 and the emitting layer 30. In the invention, the configuration of the electron-transporting zone 50 is not restricted. Specifically, although the type, the number or the like of layers are not restricted, it is normally formed of an electron-injecting layer and an electron-transporting layer.

In the invention, by using a combination of specific compounds in the hole-injecting layer 41 and the hole-transporting layer 43 constituting the hole-transporting zone 40, i.e. specifically by using a compound containing an indenofluorenedione structure in the hole-injecting layer and by using an amine compound containing a dialkylfluorene structure in the hole-transporting layer, it is possible to realize an organic EL device that can be driven at a low voltage and has a high luminous efficiency.

The thicknesses of the hole-transporting layer 43 and the hole-injecting layer 41 can be appropriately set. The thickness of each layer is normally 10 nm to 200 nm.

Hereinbelow, an explanation will be made on compounds used in the hole-injecting layer 41 and the hole-transporting layer 43.

In the present specification, a hydrogen atom includes isomers differing in number of neutrons, i.e. protium, deuterium and tritium.

In the present specification, the number of “ring carbon atoms” means the number of carbon atoms among atoms constituting a ring of a compound in which atoms are bonded in the form of a ring (for example, a monocyclic compound, a fused ring compound, a cross-linked compound, a carbocyclic compound or a heterocyclic compound). When the ring is substituted by a substituent, the carbon contained in the substituent is not included in the number of ring carbon atoms. The same is applied to the “ring carbon atoms” mentioned below, unless otherwise indicated. For example, a benzene ring includes 6 ring carbon atoms, a naphthalene ring includes 10 ring carbon atoms, a pyridinyl group includes 5 ring carbon atoms, and a furanyl group includes 4 ring carbon atoms. When a benzene ring or a naphthalene ring is substituted by an alkyl group as a substituent, for example, the number of carbon atoms of the alkyl group is not included in the number of ring carbon atoms. When a fluorene ring is bonded with a fluorene ring as a substituent (including a spirofluorene ring), for example, the number of carbon atoms of the fluorene ring as the substituent is not included in the number of ring carbon atoms.

In the present specification, the number of “ring atoms” means the number of atoms constituting a ring of a compound having a structure in which atoms are bonded in the form of a ring (for example, monocycle, fused ring, ring assembly) (for example, a monocyclic compound, a fused ring compound, a cross-linked compound, a carbocyclic compound or a heterocyclic compound). It does not include atoms which do not form a ring or atoms contained in a substituent when the ring is substituted by the substituent. The same is applied to the “ring atoms” mentioned below, unless otherwise indicated. For example, a pyridine ring includes 6 ring atoms, a quinazoline ring includes 10 ring atoms, and a furan ring includes 5 ring atoms. Hydrogen atoms respectively bonded with a carbon atom of a pyridine ring or a quinazoline ring or atoms constituting a substituent are not included in the number of ring atoms. When a fluorene ring is bonded with a fluorene ring as a substituent (including a spirofluorene ring), for example, the number of atoms of the fluorene ring as a substituent is not included in the number of ring atoms.

In the present specification, the “XX to YY carbon atoms” in the “substituted or unsubstituted ZZ group including XX to YY carbon atoms” means the number of carbon atoms when the ZZ group is unsubstituted. The number of carbon atoms of a substituent when the group is substituted is not included.

In the present specification, the “XX to YY atoms” in the “substituted or unsubstituted ZZ group including XX to YY atoms” means the number of atoms when the ZZ group is unsubstituted. The number of atoms of a substituent when the group is substituted is not included.

In the present specification, the “unsubstituted” in the “substituted or unsubstituted” means bonding of a hydrogen atom, not substitution by the substituent mentioned above.

In the invention, the hole-transporting layer comprises a compound represented by the following formula (1). Preferably, the hole-transporting layer essentially consists of this compound.

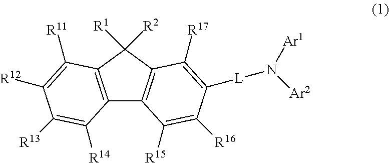

In the formula (1), R¹ and R² are independently an alkyl group including 1 to 4 carbon atoms.

R¹¹ to R¹⁷ are independently a hydrogen atom or a substituent. Adjacent ones of R¹¹ to R¹⁴ may be bonded with each other to form a ring.

Ar¹ and Ar² are independently a substituted or unsubstituted aryl group including 6 to 25 ring carbon atoms or a substituted or unsubstituted heteroaryl group including 6 to 24 ring atoms. Neither Ar¹ nor Ar² may contain a diphenylfluorene structure.

L is a single bond, a substituted or unsubstituted arylene group or a substituted or unsubstituted heteroarylene group.

It is preferred that both R¹ and R² be a methyl group.

Ar¹ and Ar² are independently a substituted or unsubstituted aryl group including 6 to 25 (preferably 6 to 24, more preferably 6 to 18) ring carbon atoms or a substituted or unsubstituted heteroaryl group including 6 to 24 (preferably 6 to 18) ring atoms.

Preferably, Ar¹ and Ar² are independently selected from the following formulas (11) to (21):

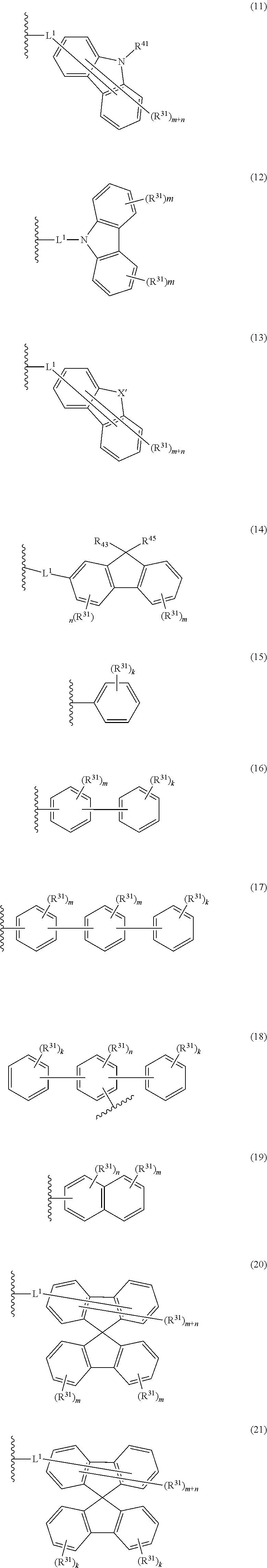

wherein in the formulas (11) to (21),

R³¹ is a substituted or unsubstituted alkyl group including 1 to 20 carbon atoms, a substituted or unsubstituted aryl group including 6 to 50 ring carbon atoms, a halogen atom, a substituted or unsubstituted fluoroalkyl group including 1 to 20 carbon atoms, a substituted or unsubstituted alkoxy group including 1 to 20 carbon atoms, a substituted or unsubstituted fluoroalkoxy group including 1 to 20 carbon atoms or a cyano group, and if plural R³¹s are present, the plural R³¹s may be the same or different;

k is an integer of 0 to 5, m is an integer of 0 to 4 and n is an integer of 0 to 3;

X′ is an oxygen atom or a sulfur atom;

L¹ is the same as L in the formula (1);

R⁴¹ is a hydrogen atom, a substituted or unsubstituted alkyl group including 1 to 20 carbon atoms, a substituted or unsubstituted aryl group including 6 to 50 ring carbon atoms, a halogen atom, a substituted or unsubstituted fluoroalkyl group including 1 to 20 carbon atoms, a substituted or unsubstituted alkoxy group including 1 to 20 carbon atoms, a substituted or unsubstituted fluoroalkoxy group including 1 to 20 carbon atoms or a cyano group;

wherein in the formula (11), R³¹ may be bonded to an arbitrary position of the carbazole skeleton, and L¹ may be bonded to an arbitrary position of the carbazole skeleton;

wherein in the formula (13), R³¹ may be bonded to an arbitrary position of the dibenzofuran skeleton or the dibenzothiophene skeleton, and L¹ may be bonded to an arbitrary position of the dibenzofuran skeleton or the dibenzothiophene skeleton;

wherein in the formula (20), R³¹ may be bonded to an arbitrary position of the fluorene skeleton, and L¹ may be bonded to an arbitrary position of the fluorene skeleton;

wherein in the formula (21), R³¹ may be bonded to an arbitrary position of the fluorene skeleton, and L¹ may be bonded to an arbitrary position of the fluorene skeleton; and

wherein in the formulas (12), (14) to (21), R³¹ may be bonded to an arbitrary position of the benzene ring, and L¹ may be bonded to an arbitrary position of the benzene ring.

R⁴³ and R⁴⁵ are independently a substituted or unsubstituted alkyl group including 1 to 20 carbon atoms, a substituted or unsubstituted aryl group including 6 to 50 ring carbon atoms, a substituted or unsubstituted heterocyclic group including 3 to 50 ring atoms, a halogen atom, a substituted or unsubstituted fluoroalkyl group including 1 to 20 carbon atoms, a substituted or unsubstituted alkoxy group including 1 to 20 carbon atoms, a substituted or unsubstituted fluoroalkoxy group including 1 to 20 carbon atoms, a substituted or unsubstituted aryl group including 6 to 50 ring carbon atoms or a cyano group, and R⁴³ and R⁴⁵ may form a ring.

Preferably, R⁴³ and R⁴⁵ are independently a substituted or unsubstituted alkyl group including 1 to 20 carbon atoms, a substituted or unsubstituted heterocyclic group including 3 to 50 ring atoms, a halogen atom, a substituted or unsubstituted fluoroalkyl group including 1 to 20 carbon atoms, a substituted or unsubstituted alkoxy group including 1 to 20 carbon atoms, a substituted or unsubstituted fluoroalkoxy group including 1 to 20 carbon atoms, a substituted or unsubstituted aryloxy group including 6 to 50 ring carbon atoms or a cyano group. R⁴³ and R⁴⁵ may forma ring.

L¹ is preferably a single bond or a phenylene group, with a single bond being more preferable.

When the compound represented by the formula (1) has plural L¹s, the plural L¹s may be the same or different.

k is any one of 0, 1, 2, 3, 4 and 5. k is preferably an integer of 0 to 3, with 1 being more preferable.

m is any one of 0, 1, 2, 3 and 4. m is preferably an integer of 0 to 3, with 1 being more preferable.

n is any one of 0, 1, 2 and 3. n is preferably an integer of 0 to 2, with 1 being more preferable.

In one embodiment of the invention, the group represented by the formula (16) and the group represented by the formula (17) may be bonded with a nitrogen atom at the para-position, the ortho-position or the meta-position, as follows.

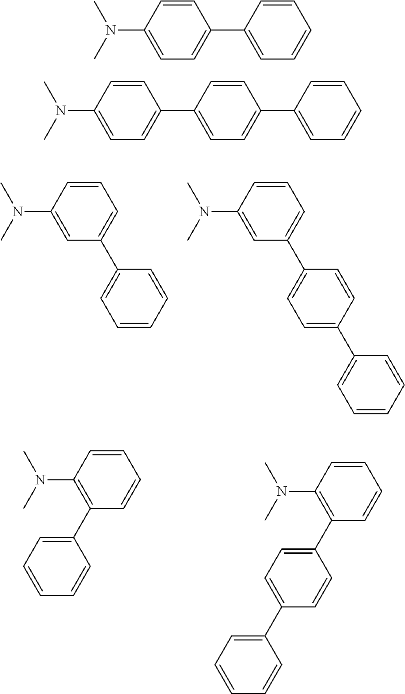

In the formula (1), Ar¹ is preferably any of groups represented by the formulas (14) to (21), more preferably a group represented by the formula (16).

When Ar¹ is a group represented by the formula (11), it is preferred that the group represented by the formula (11) be a group represented by the following formula that is bonded at the 3rd position of the carbazole skeleton.

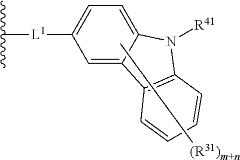

As for combinations of Ar¹ and Ar², Ar¹ and Ar² may be independently represented by any of the formulas (14) to (19), for example.

As for combinations of Ar¹ and Ar², Ar¹ and Ar² may be independently represented by any of the formulas (11) to (13), for example.

As for combinations of Ar¹ and Ar², Ar¹ and Ar² may be independently represented by any of the formulas (20) and (21), for example.

Ar¹ and Ar² may be the same.

As combinations of Ar¹ and Ar², a combination in which Ar¹ is one represented by the formula (16) and Ar² is represented by any one of formulas (14) to (21) can be given, for example.

As combinations of Ar¹ and Ar², a combination in which Ar¹ is represented by any one of formulas (14) to (19) and Ar² is represented by any one of formulas (11) to (13) can be given, for example.

L is preferably a single bond, a substituted or unsubstituted arylene group including 6 to 24 (preferably 6 to 18) ring carbon atoms or a substituted or unsubstituted heteroarylene group including 6 to 24 (preferably 6 to 18) ring atoms. L is more preferably a single bond or a substituted or unsubstituted arylene group.

L is preferably a group represented by the following formula (22):

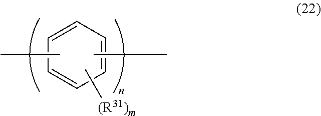

In the formula (22), R³¹ is a substituted or unsubstituted alkyl group including 1 to 20 carbon atoms, a substituted or unsubstituted aryl group including 6 to 50 ring carbon atoms, a halogen atom, a substituted or unsubstituted fluoroalkyl group including 1 to 20 carbon atoms, a substituted or unsubstituted alkoxy group including 1 to 20 carbon atoms, a substituted or unsubstituted fluoroalkoxy group including 1 to 20 carbon atoms or a cyano group. When plural R³¹s are present, the plural R³¹s may be the same or different.

m is an integer of 0 to 4. n is an integer of 0 to 3.

When n is 0, L is a single bond. When n is plural, the plural R³¹s may be the same or different and the plural ms may be the same or different.

n is preferably 0 to 2, with 0 being more preferable.

It is preferred that n benzene rings be bonded with each other at the para-position.

The substituent represented by R¹¹ to R¹⁷ is preferably one or more selected from a substituted or unsubstituted aryl group including 6 to 50 ring carbon atoms, a substituted or unsubstituted alkyl group including 1 to 20 carbon atoms, a substituted or unsubstituted alkoxy group including 1 to 20 carbon atoms, a substituted or unsubstituted aryloxy group including 6 to 50 ring carbon atoms, a substituted or unsubstituted arylthio group including 6 to 50 ring atoms, a substituted or unsubstituted alkoxycarbonyl group including 2 to 50 carbon atoms, an amino group substituted by a substituted or unsubstituted aryl group including 6 to 50 ring carbon atoms, a halogen atom, a cyano group, a nitro group, a hydroxyl group and a carboxy group.

In a preferred embodiment of the invention, as R¹¹ to R¹⁷, a hydrogen atom, a halogen atom (in particular, a fluorine atom), an alkyl group (in particular, a methyl group, a t-butyl group) and an aryl group (in particular, a phenyl group) are preferable.

In the formula (1), the substituent of the “substituted or unsubstituted” of Ar¹, Ar² and L is preferably one or more selected from a substituted or unsubstituted aryl group including 6 to 50 ring carbon atoms, a substituted or unsubstituted alkyl group including 1 to 20 carbon atoms, a substituted or unsubstituted alkoxy group including 1 to 20 carbon atoms, a substituted or unsubstituted aryloxy group including 6 to 50 ring carbon atoms, a substituted or unsubstituted arylthio group including 6 to 50 ring atoms, a substituted or unsubstituted alkoxycarbonyl group including 2 to 50 carbon atoms, an amino group substituted by a substituted or unsubstituted aryl group including 6 to 50 ring carbon atoms, a halogen atom, a cyano group, a nitro group, a hydroxyl group and a carboxyl group.

Specific examples of the compounds represented by the formula (1) are given below. However, the compounds represented by the formula (1) are not limited thereto.

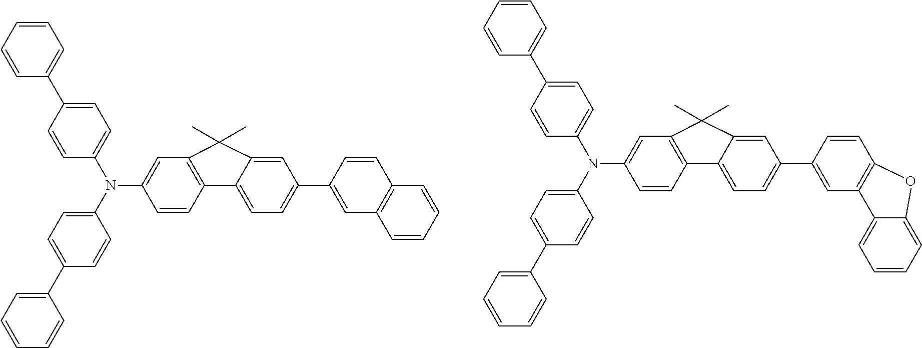 [SMILES: CC1(C)c2cc(-c3ccc4ccccc4c3)ccc2-c2ccc(N(c3ccc(-c4ccccc4)cc3)c3ccc(-c4ccccc4)cc3)cc21 ; CC1(C)c2cc(-c3ccc4oc5ccccc5c4c3)ccc2-c2ccc(N(c3ccc(-c4ccccc4)cc3)c3ccc(-c4ccccc4)cc3)cc21]

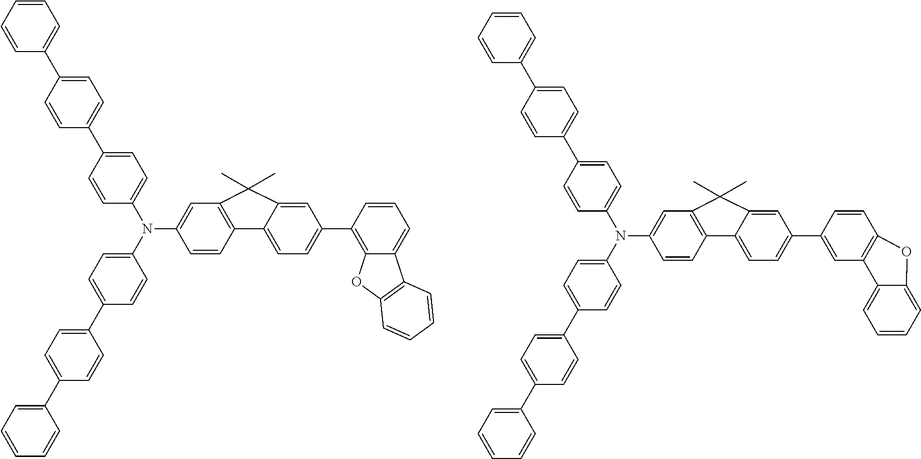 [SMILES: CC1(C)c2cc(-c3cccc4c3oc3ccccc34)ccc2-c2ccc(N(c3ccc(-c4ccc(-c5ccccc5)cc4)cc3)c3ccc(-c4ccc(-c5ccccc5)cc4)cc3)cc21 ; CC1(C)c2cc(-c3ccc4oc5ccccc5c4c3)ccc2-c2ccc(N(c3ccc(-c4ccc(-c5ccccc5)cc4)cc3)c3ccc(-c4ccc(-c5ccccc5)cc4)cc3)cc21]

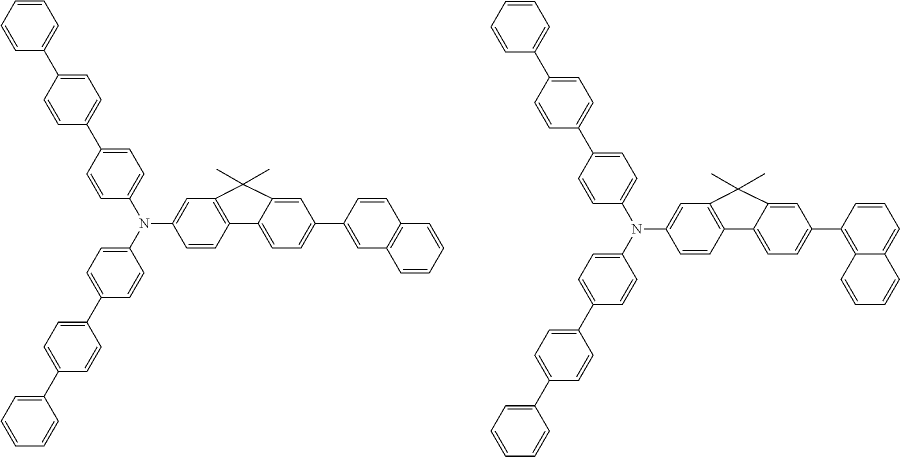 [SMILES: CC1(C)c2cc(-c3ccc4ccccc4c3)ccc2-c2ccc(N(c3ccc(-c4ccc(-c5ccccc5)cc4)cc3)c3ccc(-c4ccc(-c5ccccc5)cc4)cc3)cc21 ; CC1(C)c2cc(-c3cccc4ccccc34)ccc2-c2ccc(N(c3ccc(-c4ccc(-c5ccccc5)cc4)cc3)c3ccc(-c4ccc(-c5ccccc5)cc4)cc3)cc21]

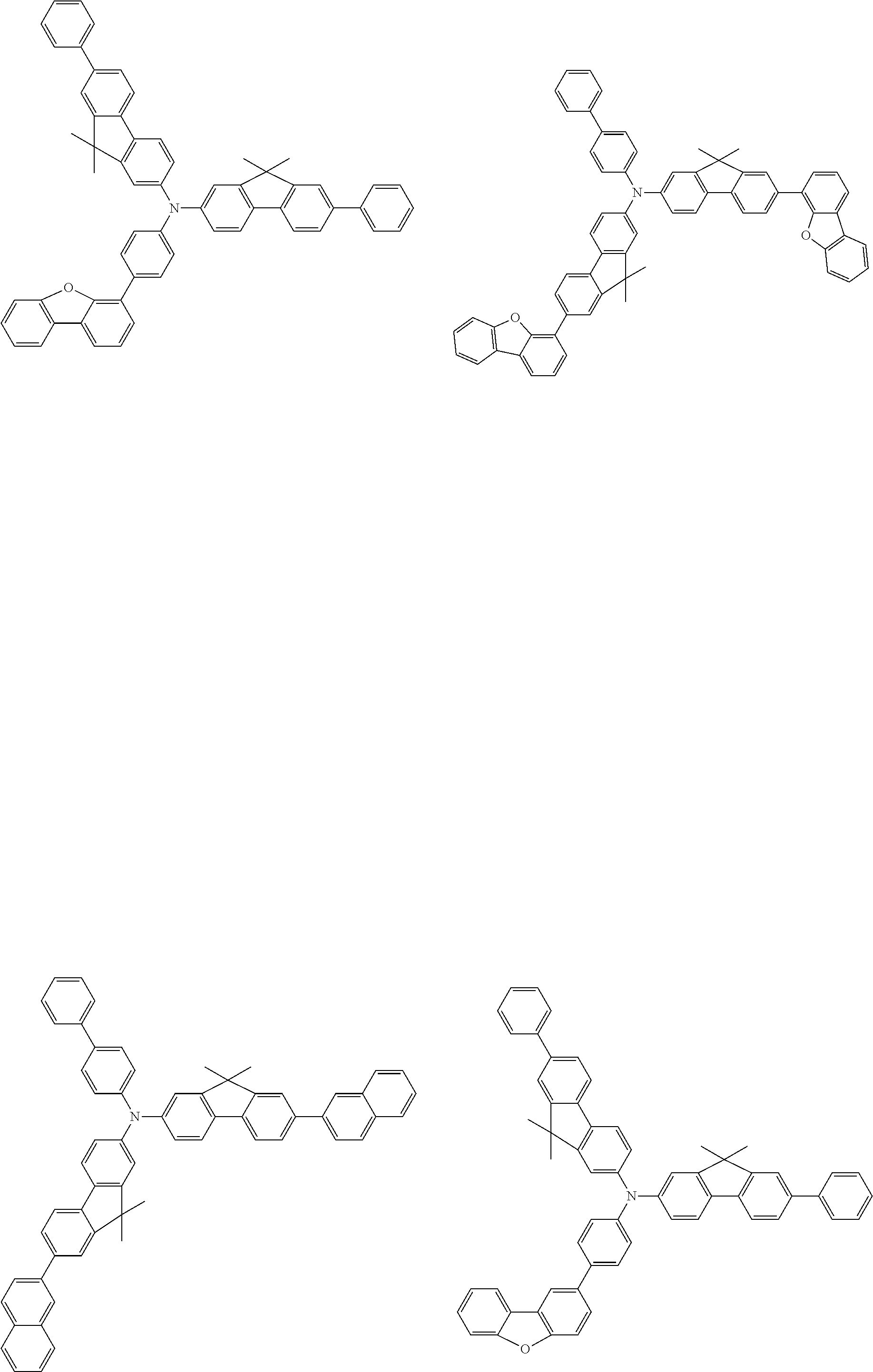 [SMILES: CC1(C)c2cc(-c3ccccc3)ccc2-c2ccc(N(c3ccc(-c4cccc5c4oc4ccccc45)cc3)c3ccc4c(c3)C(C)(C)c3cc(-c5ccccc5)ccc3-4)cc21 ; CC1(C)c2cc(-c3cccc4c3oc3ccccc34)ccc2-c2ccc(N(c3ccc(-c4ccccc4)cc3)c3ccc4c(c3)C(C)(C)c3cc(-c5cccc6c5oc5ccccc56)ccc3-4)cc21 ; CC1(C)c2cc(-c3ccc4ccccc4c3)ccc2-c2ccc(N(c3ccc(-c4ccccc4)cc3)c3ccc4c(c3)C(C)(C)c3cc(-c5ccc6ccccc6c5)ccc3-4)cc21 ; CC1(C)c2cc(-c3ccccc3)ccc2-c2ccc(N(c3ccc(-c4ccc5oc6ccccc6c5c4)cc3)c3ccc4c(c3)C(C)(C)c3cc(-c5ccccc5)ccc3-4)cc21]

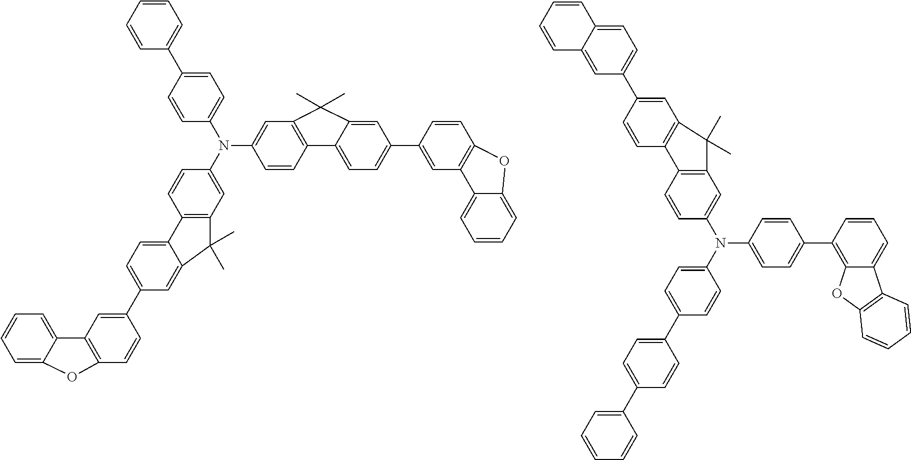 [SMILES: CC1(C)c2cc(-c3ccc4oc5ccccc5c4c3)ccc2-c2ccc(N(c3ccc(-c4ccccc4)cc3)c3ccc4c(c3)C(C)(C)c3cc(-c5ccc6oc7ccccc7c6c5)ccc3-4)cc21 ; CC1(C)c2cc(-c3ccc4ccccc4c3)ccc2-c2ccc(N(c3ccc(-c4ccc(-c5ccccc5)cc4)cc3)c3ccc(-c4cccc5c4oc4ccccc45)cc3)cc21]

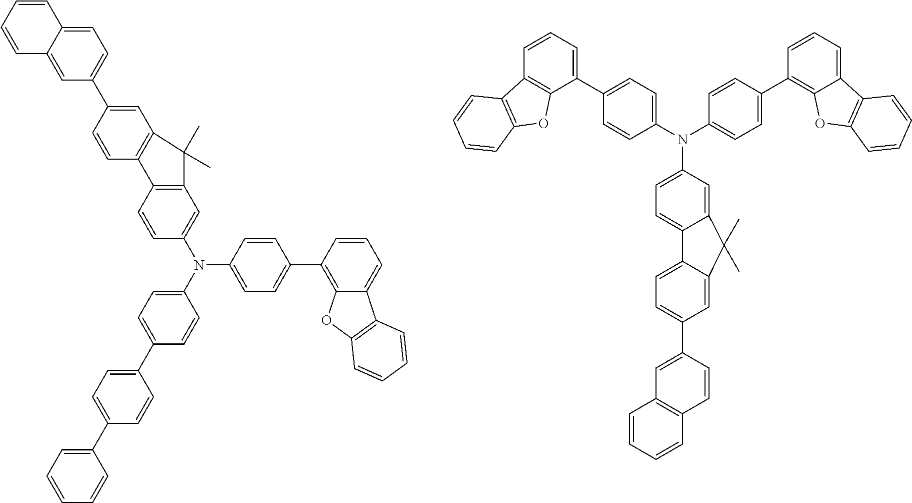 [SMILES: CC1(C)c2cc(-c3ccc4ccccc4c3)ccc2-c2ccc(N(c3ccc(-c4ccc(-c5ccccc5)cc4)cc3)c3ccc(-c4cccc5c4oc4ccccc45)cc3)cc21 ; CC1(C)c2cc(-c3ccc4ccccc4c3)ccc2-c2ccc(N(c3ccc(-c4cccc5c4oc4ccccc45)cc3)c3ccc(-c4cccc5c4oc4ccccc45)cc3)cc21]

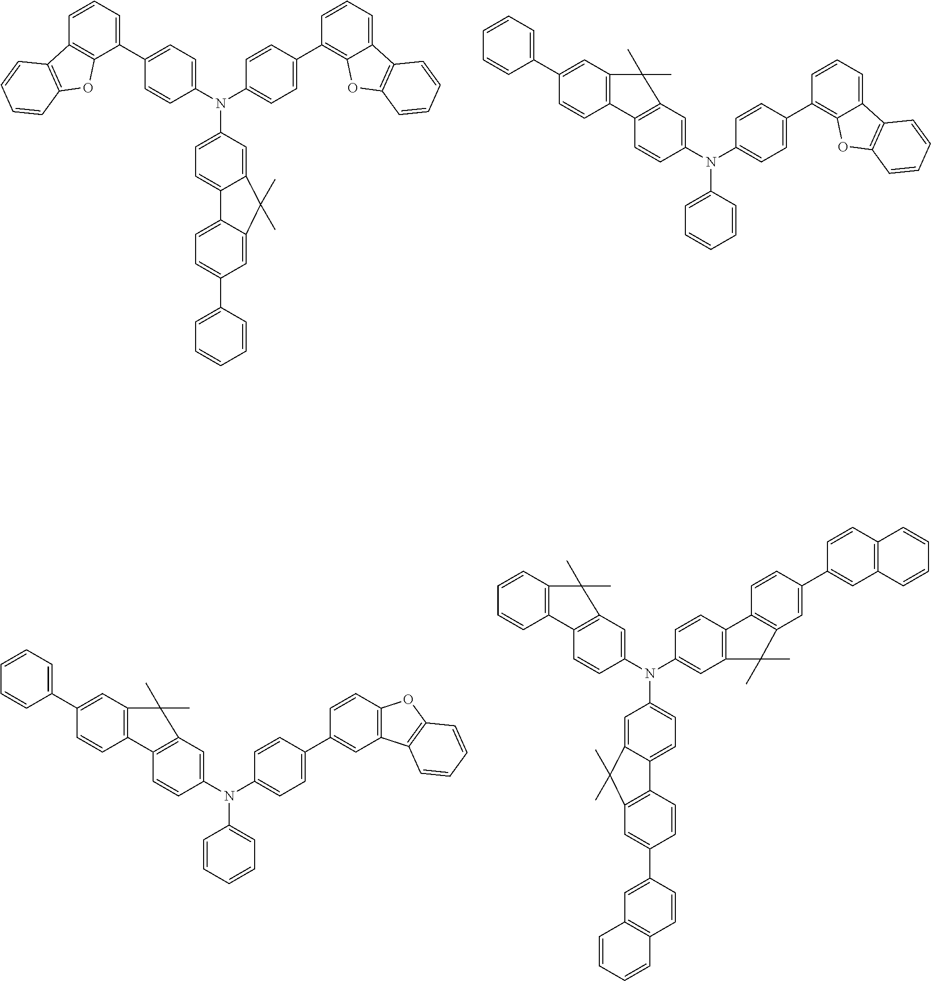 [SMILES: CC1(C)c2cc(-c3ccccc3)ccc2-c2ccc(N(c3ccc(-c4cccc5c4oc4ccccc45)cc3)c3ccc(-c4cccc5c4oc4ccccc45)cc3)cc21 ; CC1(C)c2cc(-c3ccccc3)ccc2-c2ccc(N(c3ccccc3)c3ccc(-c4cccc5c4oc4ccccc45)cc3)cc21 ; CC1(C)c2cc(-c3ccccc3)ccc2-c2ccc(N(c3ccccc3)c3ccc(-c4ccc5oc6ccccc6c5c4)cc3)cc21 ; CC1(C)c2ccccc2-c2ccc(N(c3ccc4c(c3)C(C)(C)c3cc(-c5ccc6ccccc6c5)ccc3-4)c3ccc4c(c3)C(C)(C)c3cc(-c5ccc6ccccc6c5)ccc3-4)cc21]

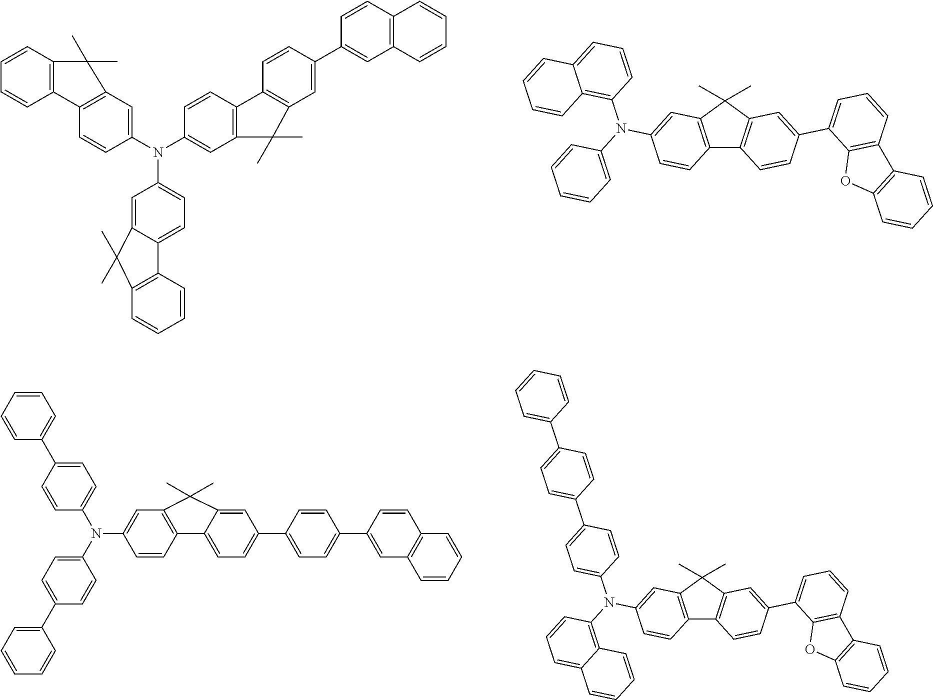 [SMILES: CC1(C)c2ccccc2-c2ccc(N(c3ccc4c(c3)C(C)(C)c3ccccc3-4)c3ccc4c(c3)C(C)(C)c3cc(-c5ccc6ccccc6c5)ccc3-4)cc21 ; CC1(C)c2cc(-c3cccc4c3oc3ccccc34)ccc2-c2ccc(N(c3ccccc3)c3cccc4ccccc34)cc21 ; CC1(C)c2cc(-c3ccc(-c4ccc5ccccc5c4)cc3)ccc2-c2ccc(N(c3ccc(-c4ccccc4)cc3)c3ccc(-c4ccccc4)cc3)cc21 ; CC1(C)c2cc(-c3cccc4c3oc3ccccc34)ccc2-c2ccc(N(c3ccc(-c4ccc(-c5ccccc5)cc4)cc3)c3cccc4ccccc34)cc21]

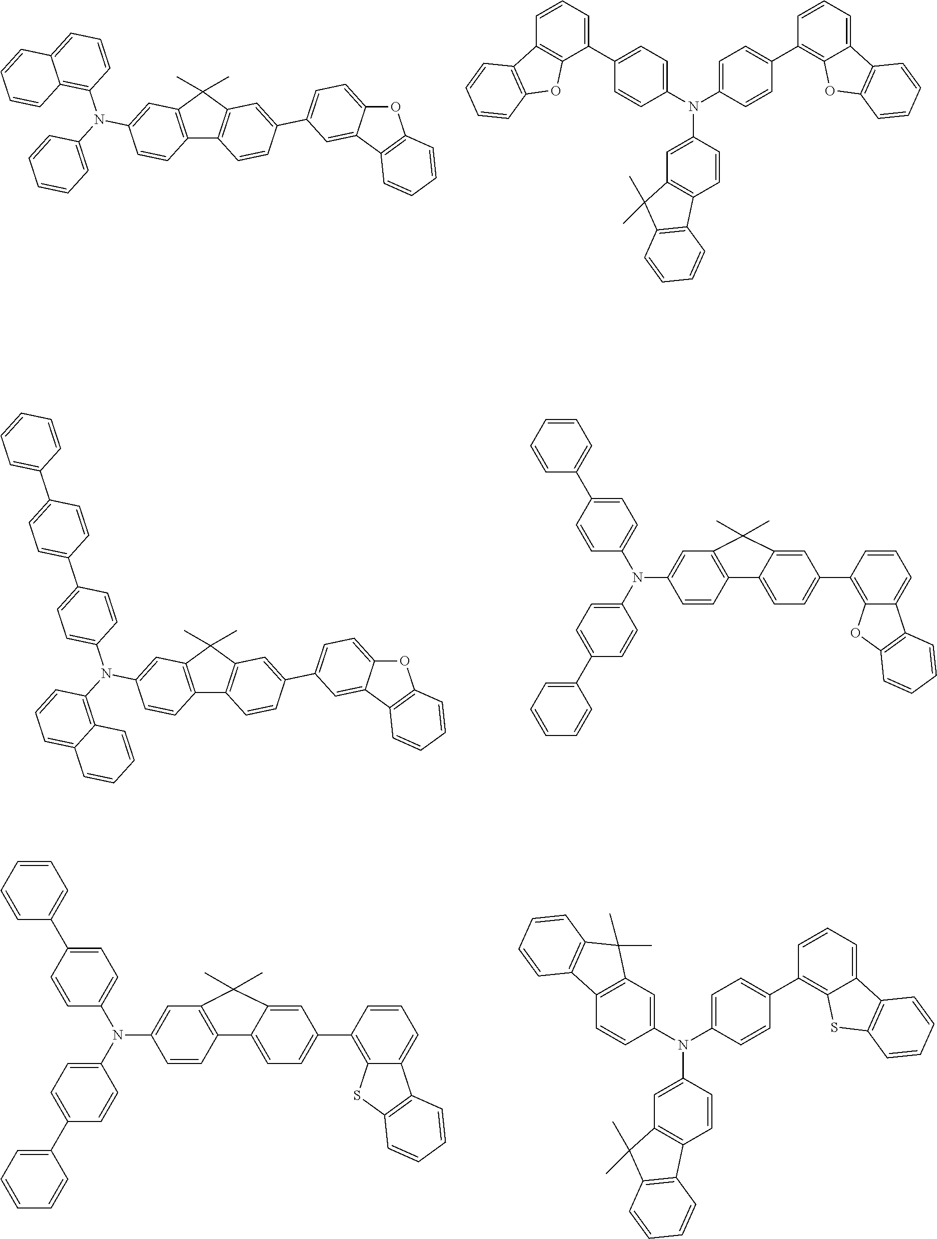

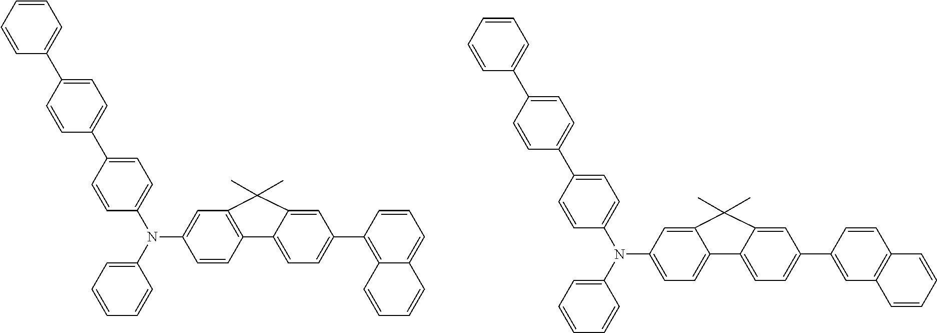 [SMILES: CC1(C)c2cc(-c3cccc4ccccc34)ccc2-c2ccc(N(c3ccccc3)c3ccc(-c4ccc(-c5ccccc5)cc4)cc3)cc21 ; CC1(C)c2cc(-c3ccc4ccccc4c3)ccc2-c2ccc(N(c3ccccc3)c3ccc(-c4ccc(-c5ccccc5)cc4)cc3)cc21]

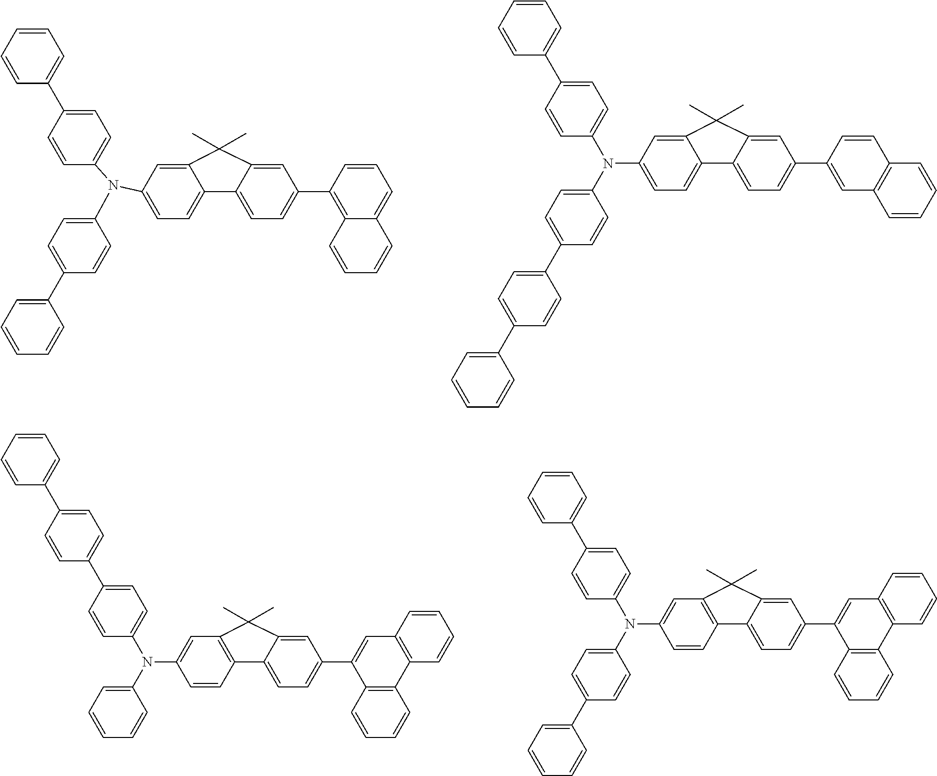 [SMILES: CC1(C)c2cc(-c3cccc4ccccc34)ccc2-c2ccc(N(c3ccc(-c4ccccc4)cc3)c3ccc(-c4ccccc4)cc3)cc21 ; CC1(C)c2cc(-c3ccc4ccccc4c3)ccc2-c2ccc(N(c3ccc(-c4ccccc4)cc3)c3ccc(-c4ccc(-c5ccccc5)cc4)cc3)cc21 ; CC1(C)c2cc(-c3cc4ccccc4c4ccccc34)ccc2-c2ccc(N(c3ccccc3)c3ccc(-c4ccc(-c5ccccc5)cc4)cc3)cc21 ; CC1(C)c2cc(-c3cc4ccccc4c4ccccc34)ccc2-c2ccc(N(c3ccc(-c4ccccc4)cc3)c3ccc(-c4ccccc4)cc3)cc21]

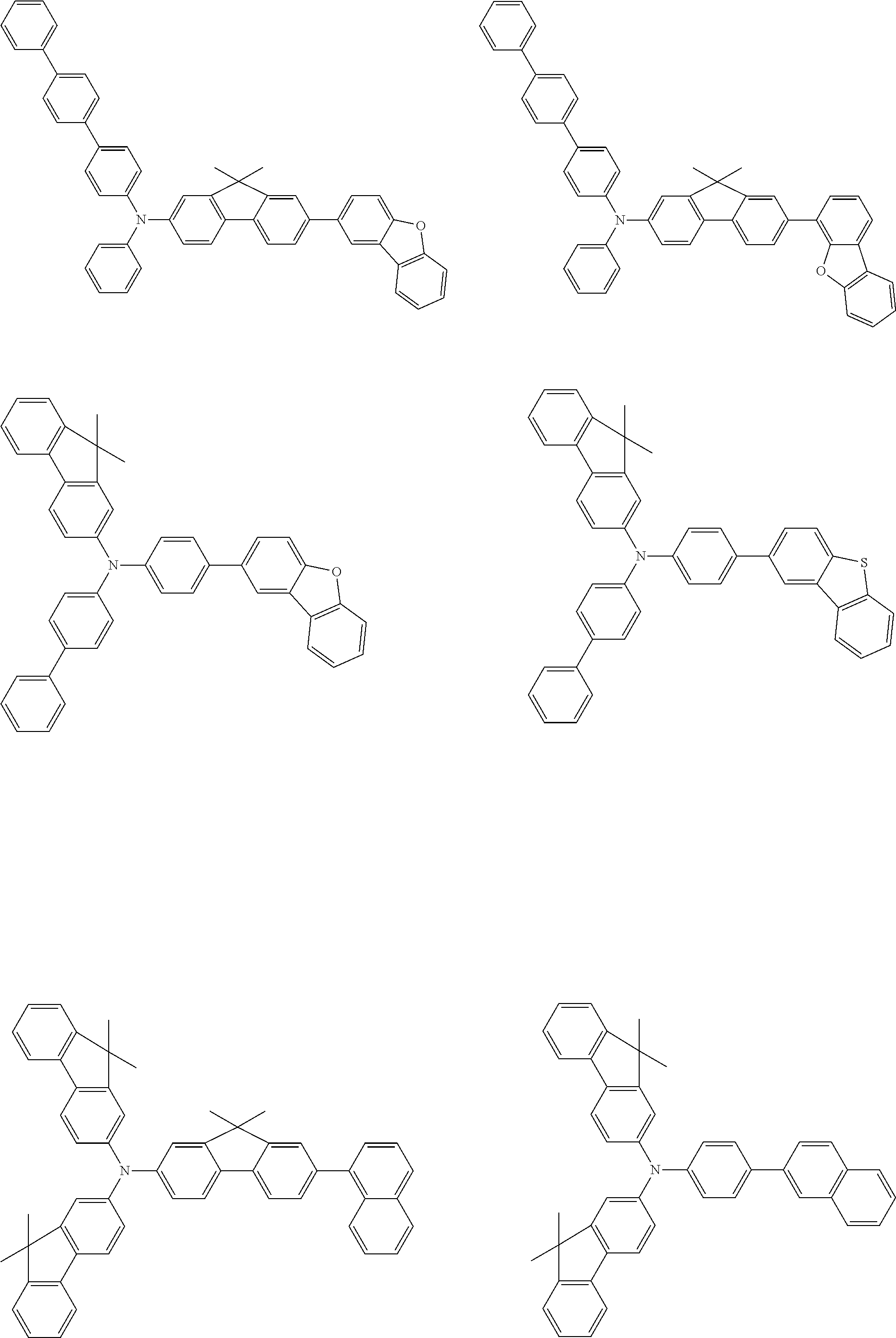

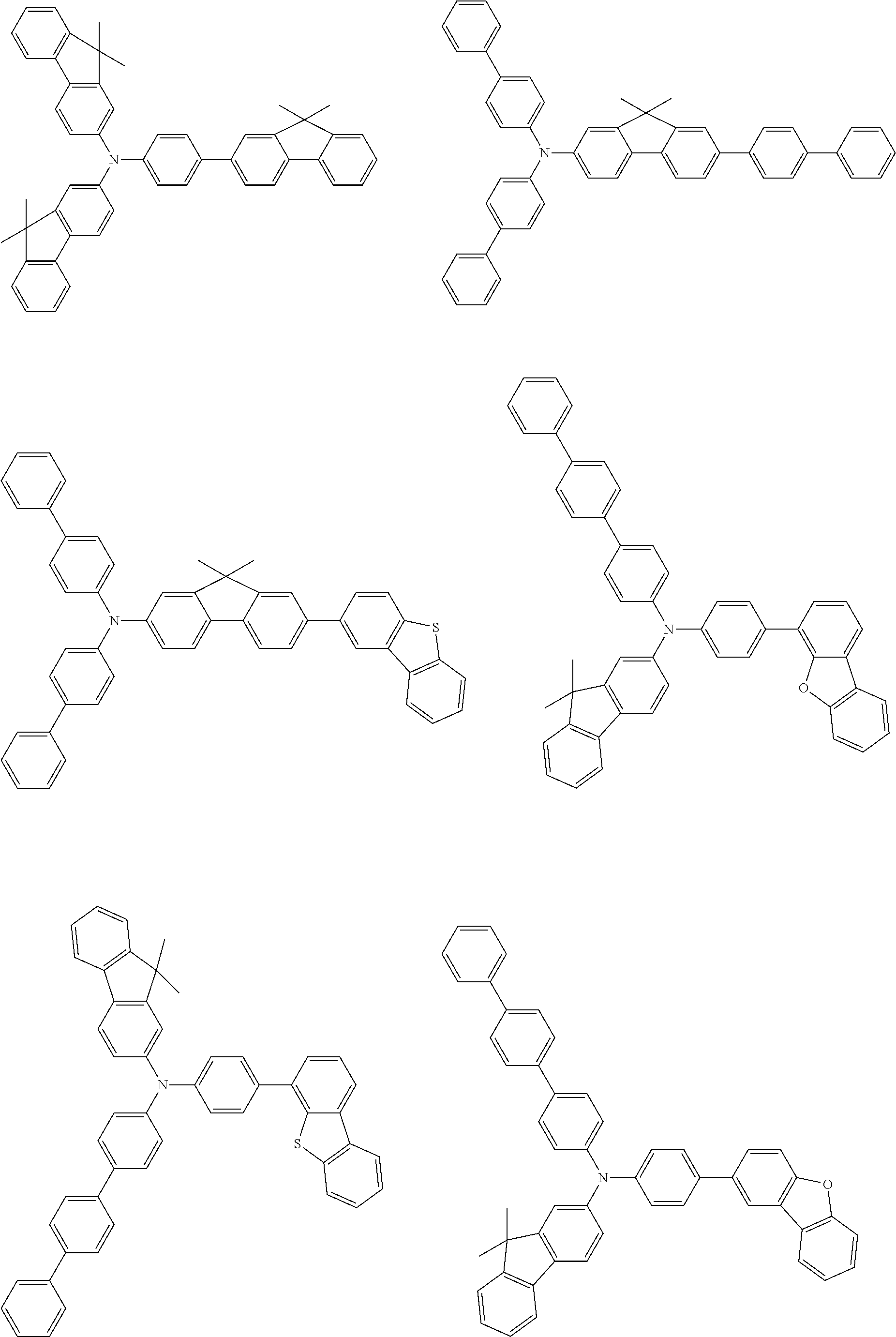

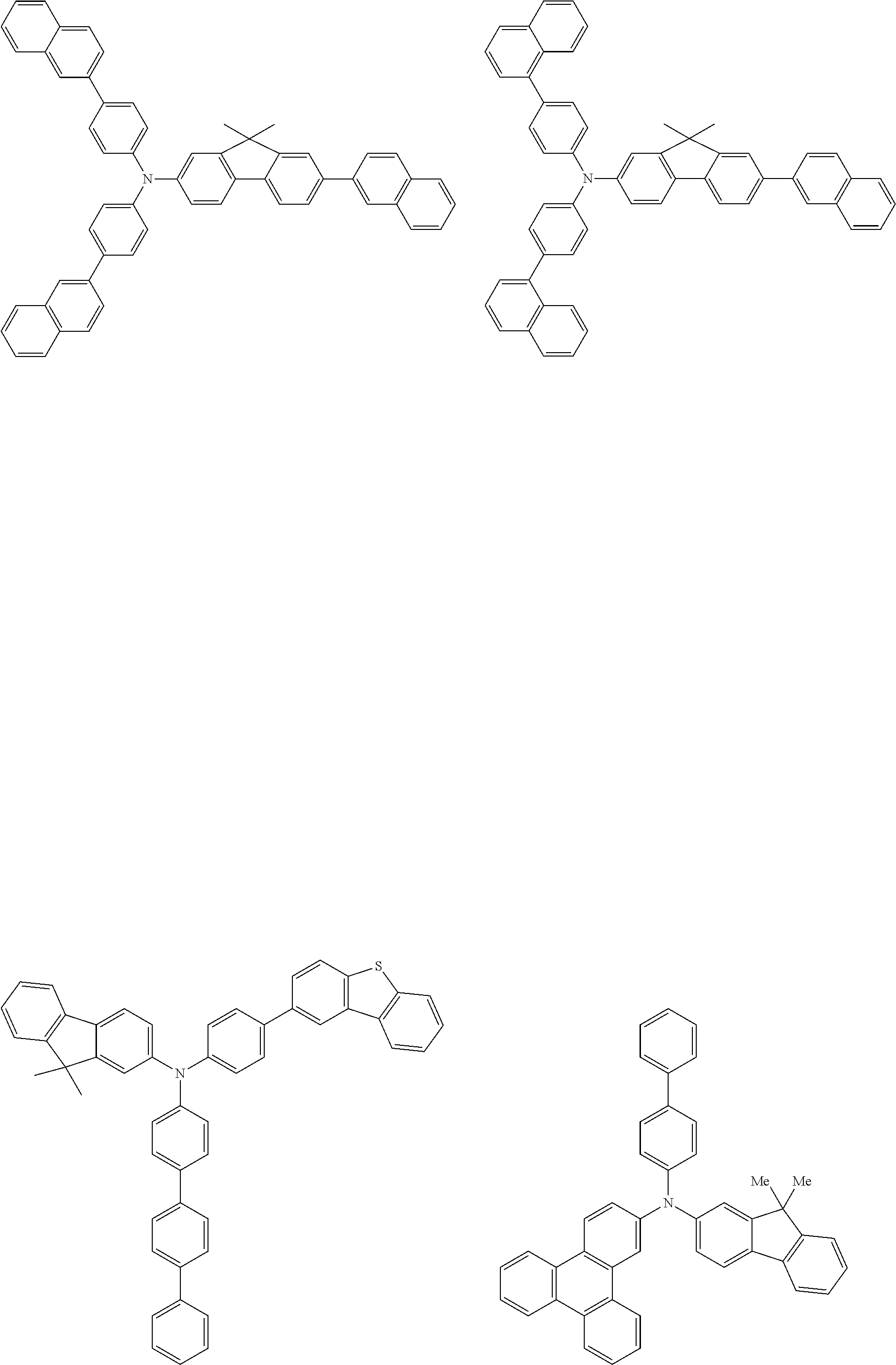 [SMILES: CC1(C)c2cc(-c3ccc4ccccc4c3)ccc2-c2ccc(N(c3ccc(-c4ccc5ccccc5c4)cc3)c3ccc(-c4ccc5ccccc5c4)cc3)cc21 ; CC1(C)c2cc(-c3ccc4ccccc4c3)ccc2-c2ccc(N(c3ccc(-c4cccc5ccccc45)cc3)c3ccc(-c4cccc5ccccc45)cc3)cc21 ; CC1(C)c2ccccc2-c2ccc(N(c3ccc(-c4ccc(-c5ccccc5)cc4)cc3)c3ccc(-c4ccc5sc6ccccc6c5c4)cc3)cc21 ; CC1(C)c2ccccc2-c2ccc(N(c3ccc(-c4ccccc4)cc3)c3ccc4c5ccccc5c5ccccc5c4c3)cc21]

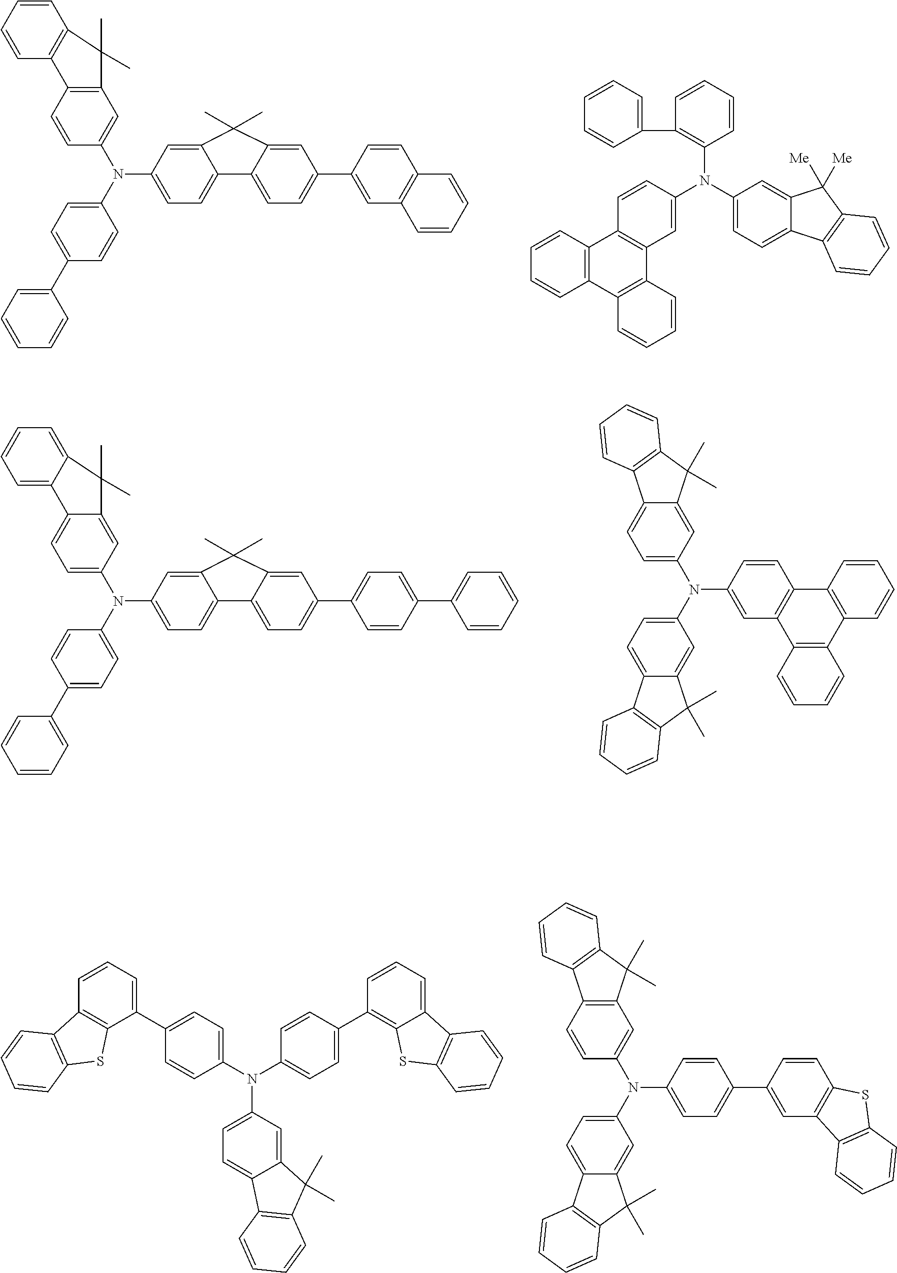

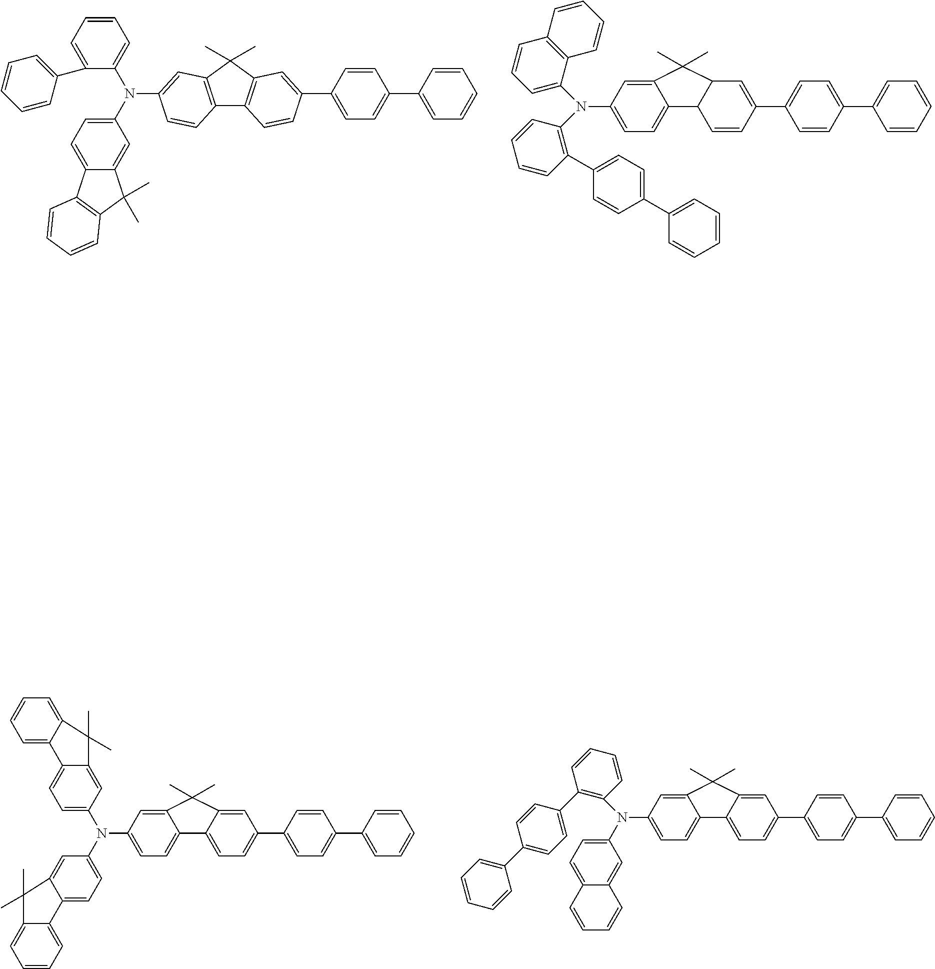

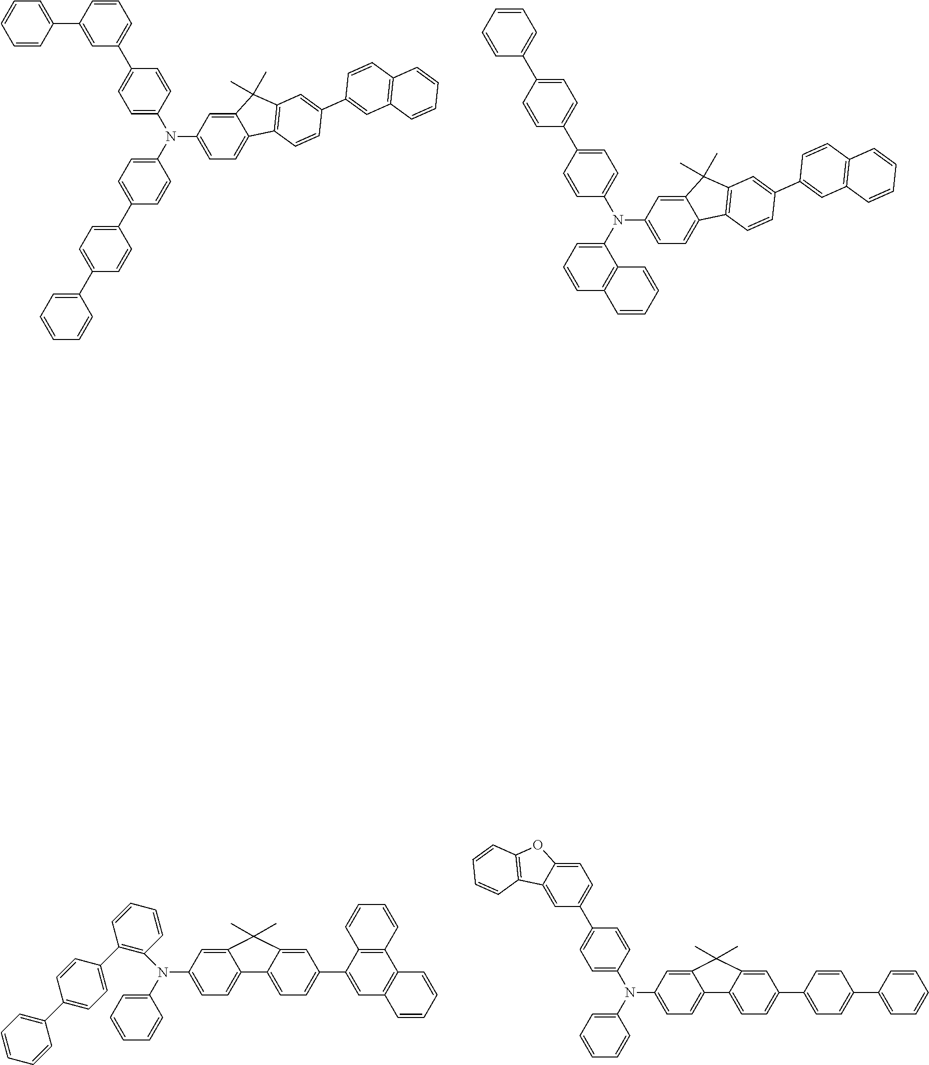 [SMILES: CC1(C)c2cc(-c3ccc4ccccc4c3)ccc2-c2ccc(N(c3ccc(-c4ccc(-c5ccccc5)cc4)cc3)c3ccc(-c4cccc(-c5ccccc5)c4)cc3)cc21 ; CC1(C)c2cc(-c3ccc4ccccc4c3)ccc2-c2ccc(N(c3ccc(-c4ccc(-c5ccccc5)cc4)cc3)c3cccc4ccccc34)cc21 ; CC1(C)c2cc(-c3cc4ccccc4c4ccccc34)ccc2-c2ccc(N(c3ccccc3)c3ccccc3-c3ccc(-c4ccccc4)cc3)cc21 ; CC1(C)c2cc(-c3ccc(-c4ccccc4)cc3)ccc2-c2ccc(N(c3ccccc3)c3ccc(-c4ccc5oc6ccccc6c5c4)cc3)cc21]

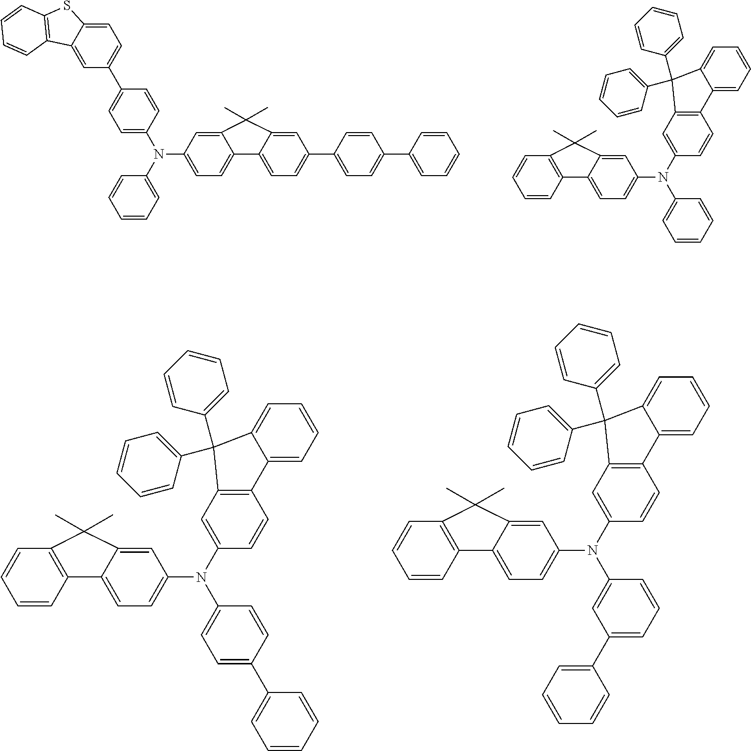 [SMILES: CC1(C)c2cc(-c3ccc(-c4ccccc4)cc3)ccc2-c2ccc(N(c3ccccc3)c3ccc(-c4ccc5sc6ccccc6c5c4)cc3)cc21 ; CC1(C)c2ccccc2-c2ccc(N(c3ccccc3)c3ccc4c(c3)C(c3ccccc3)(c3ccccc3)c3ccccc3-4)cc21 ; CC1(C)c2ccccc2-c2ccc(N(c3ccc(-c4ccccc4)cc3)c3ccc4c(c3)C(c3ccccc3)(c3ccccc3)c3ccccc3-4)cc21 ; CC1(C)c2ccccc2-c2ccc(N(c3cccc(-c4ccccc4)c3)c3ccc4c(c3)C(c3ccccc3)(c3ccccc3)c3ccccc3-4)cc21]

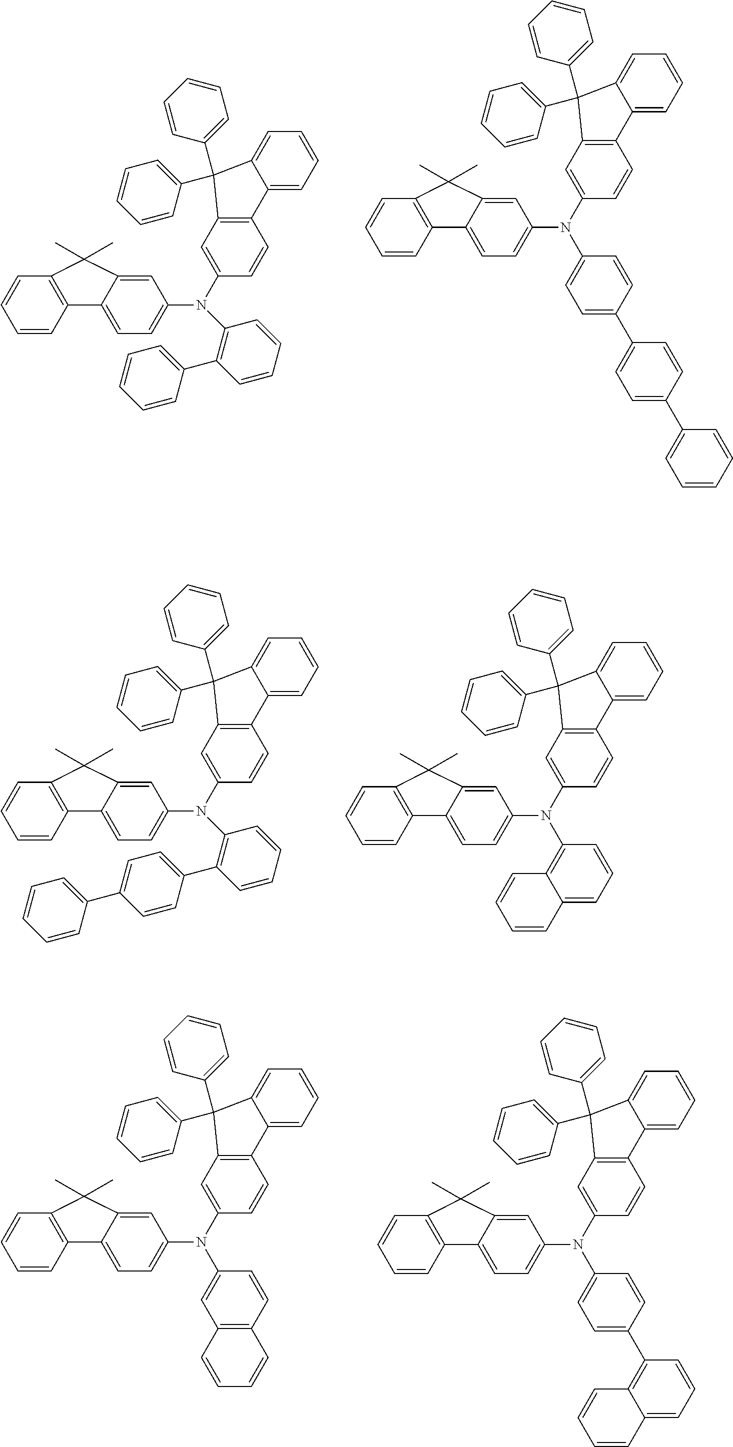

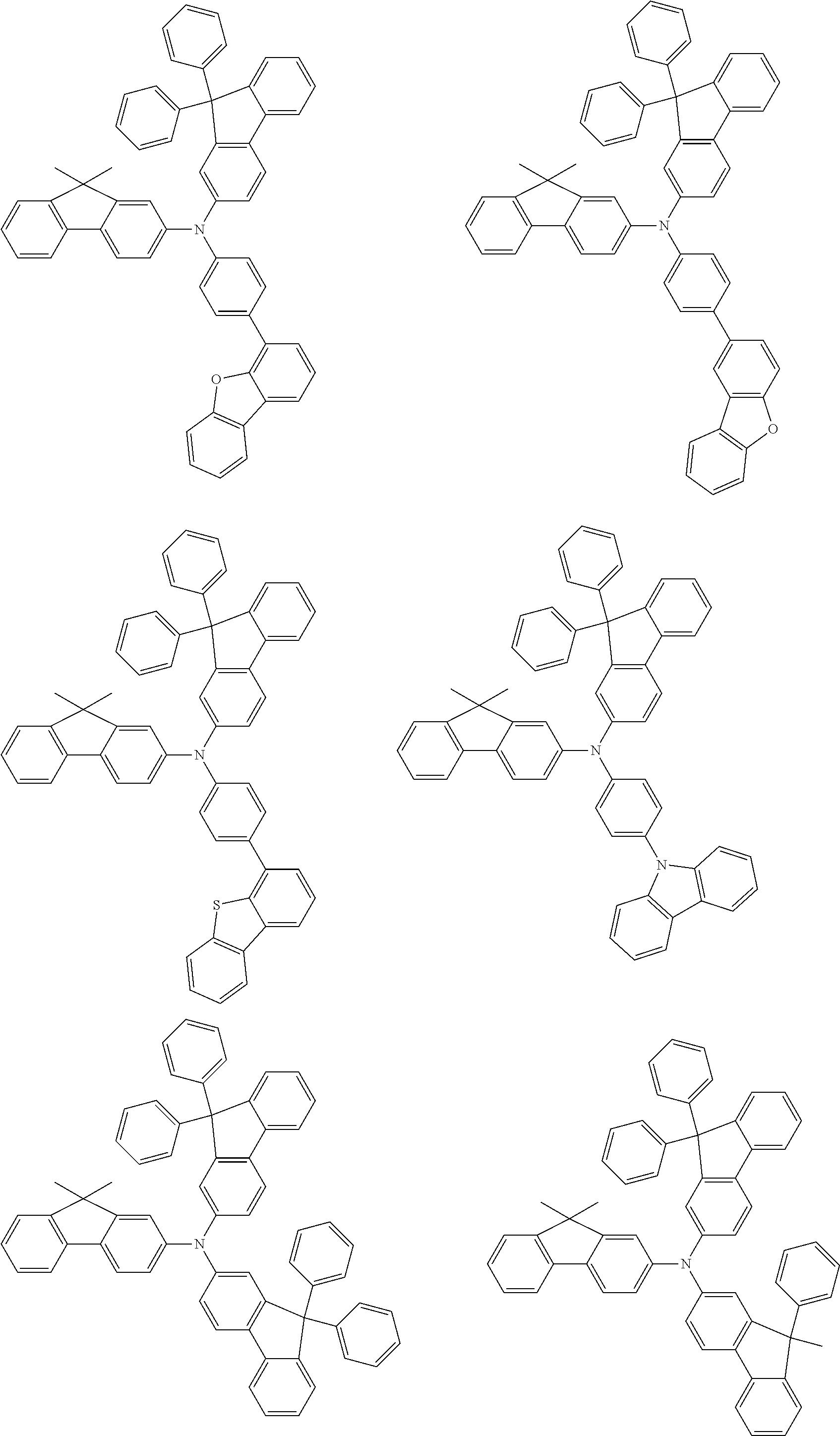

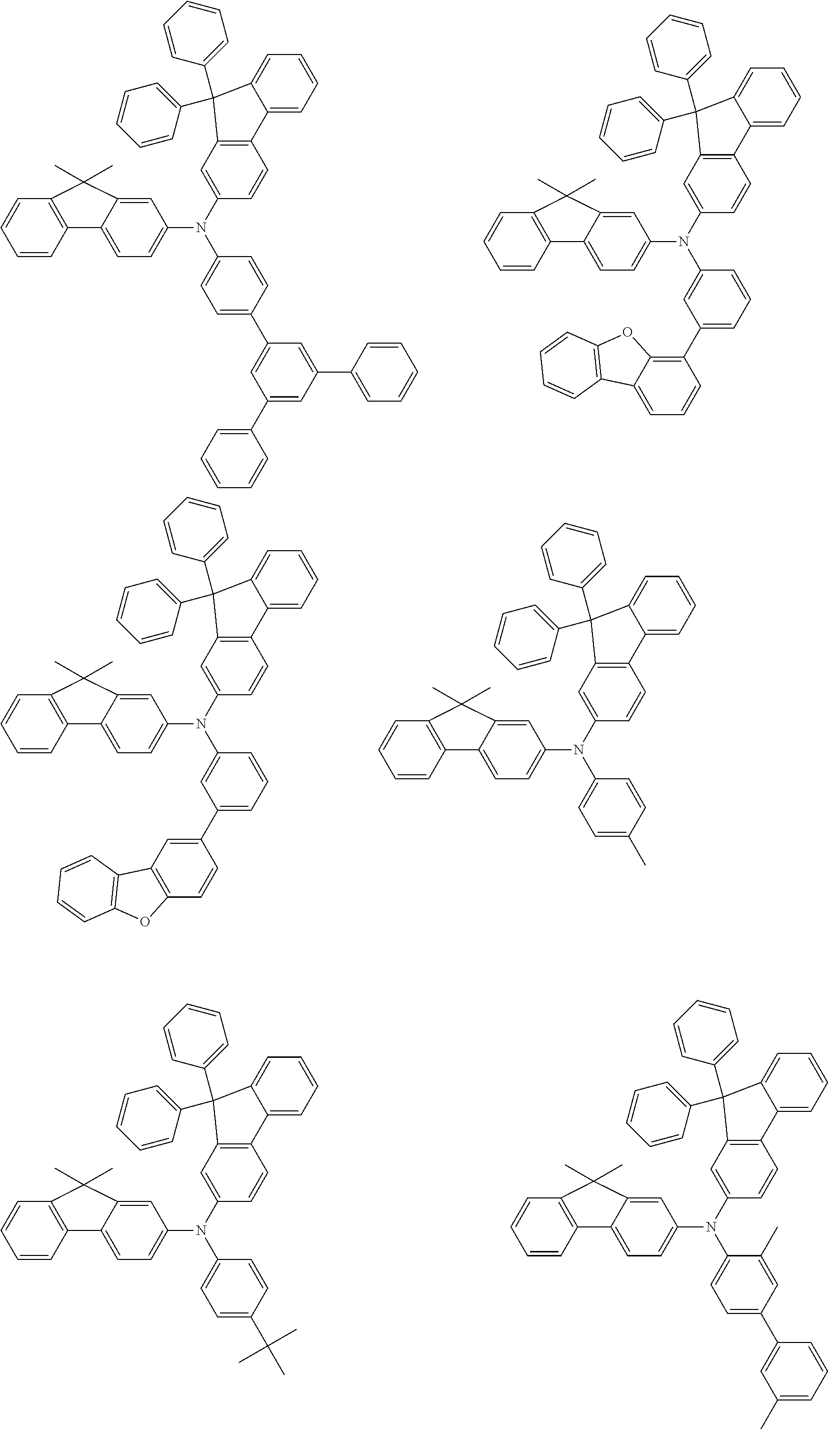

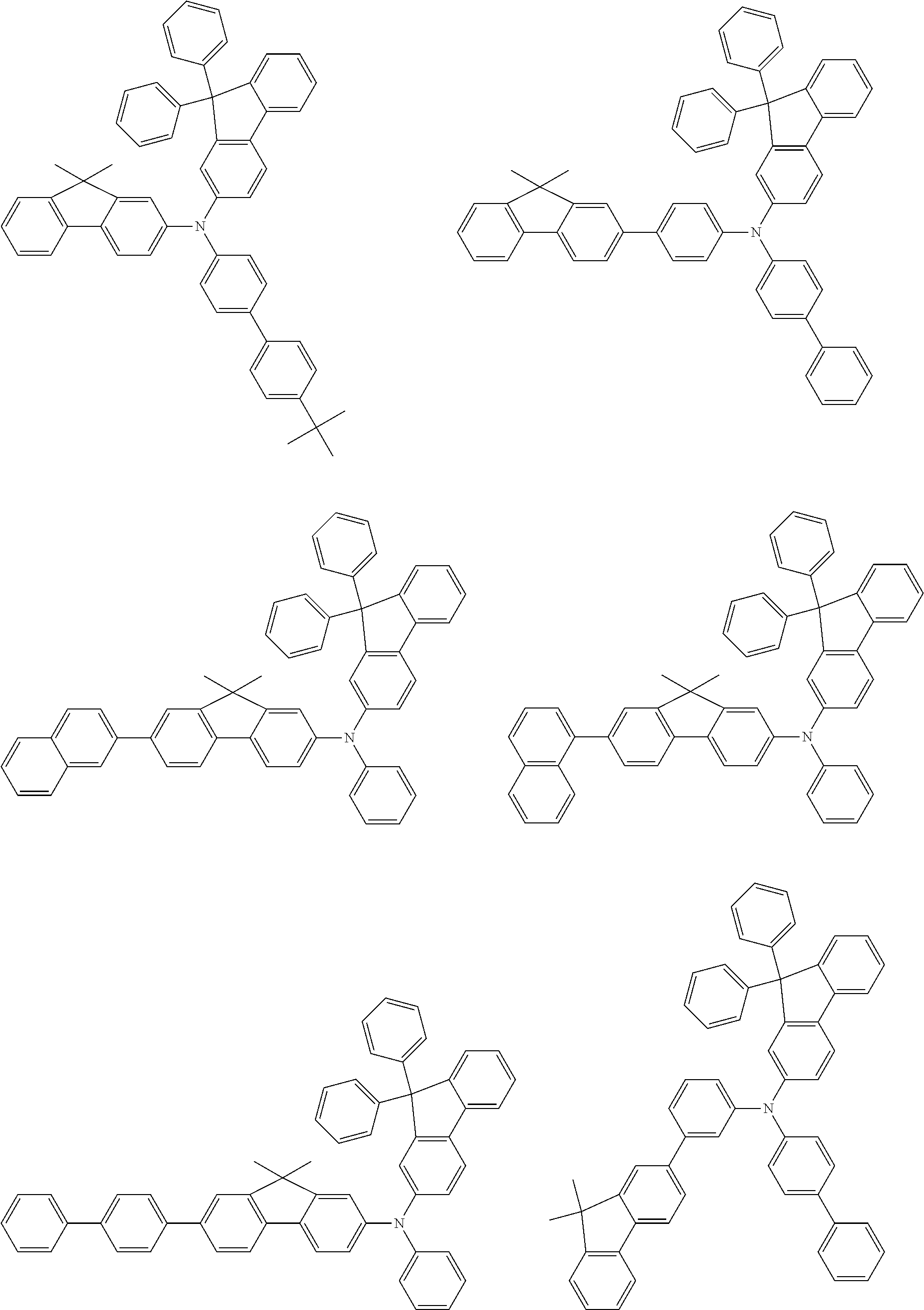

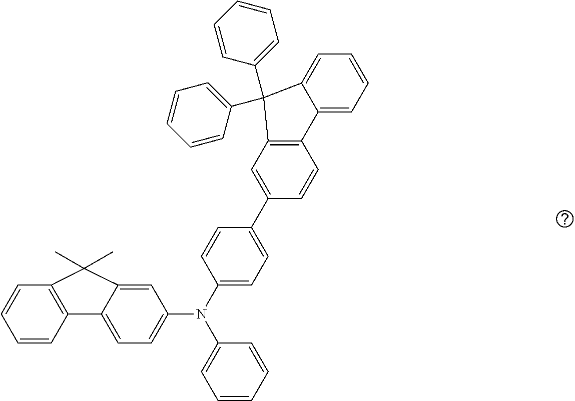 [SMILES: CC1(C)c2ccccc2-c2ccc(N(c3ccccc3)c3ccc(-c4ccc5c(c4)C(c4ccccc4)(c4ccccc4)c4ccccc4-5)cc3)cc21]

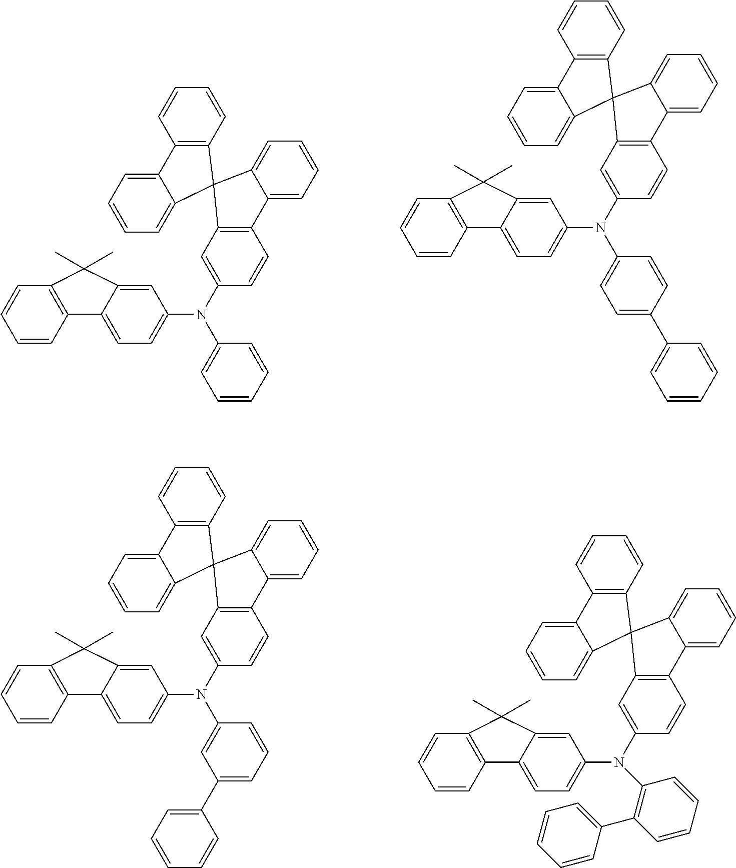 [SMILES: CC1(C)c2ccccc2-c2ccc(N(c3ccccc3)c3ccc4c(c3)C3(c5ccccc5-c5ccccc53)c3ccccc3-4)cc21 ; CC1(C)c2ccccc2-c2ccc(N(c3ccc(-c4ccccc4)cc3)c3ccc4c(c3)C3(c5ccccc5-c5ccccc53)c3ccccc3-4)cc21 ; CC1(C)c2ccccc2-c2ccc(N(c3cccc(-c4ccccc4)c3)c3ccc4c(c3)C3(c5ccccc5-c5ccccc53)c3ccccc3-4)cc21 ; CC1(C)c2ccccc2-c2ccc(N(c3ccc4c(c3)C3(c5ccccc5-c5ccccc53)c3ccccc3-4)c3ccccc3-c3ccccc3)cc21]

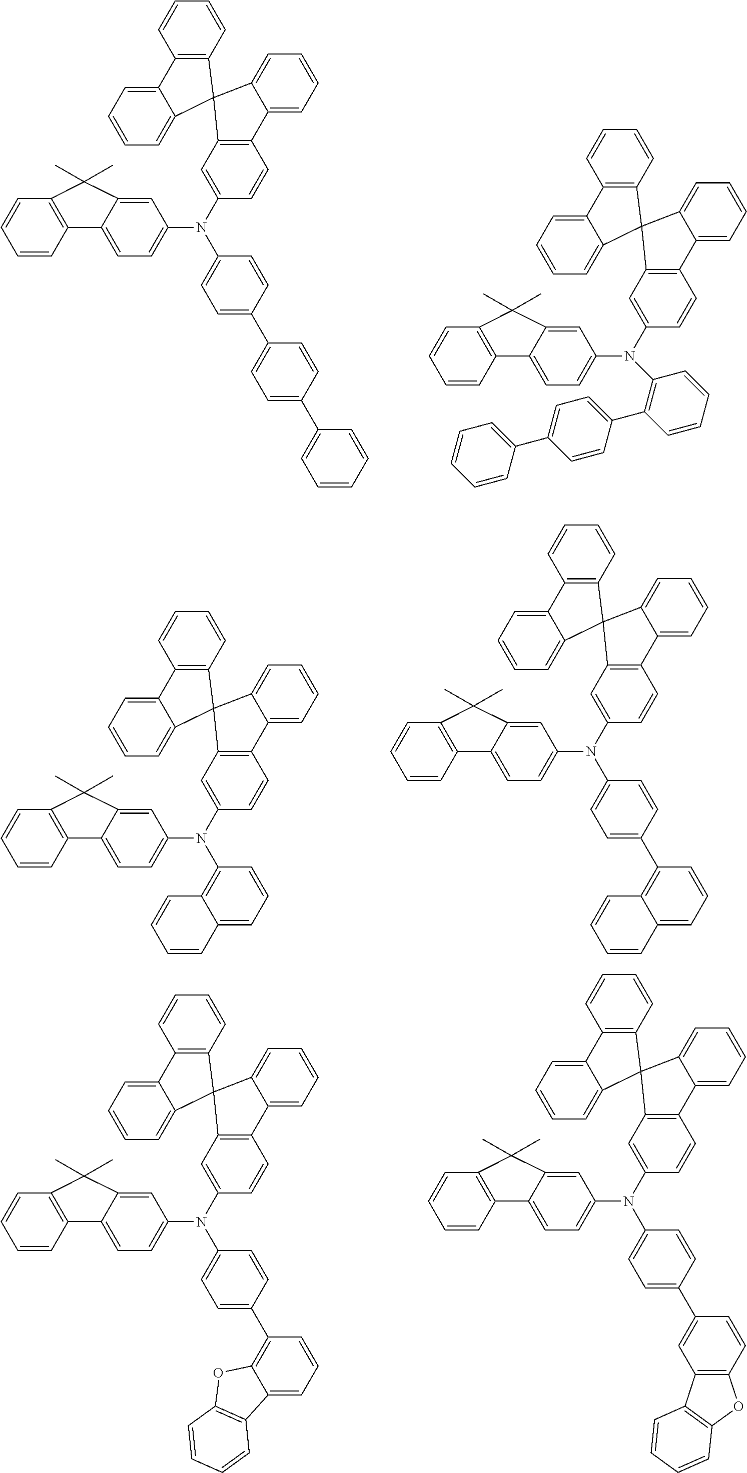

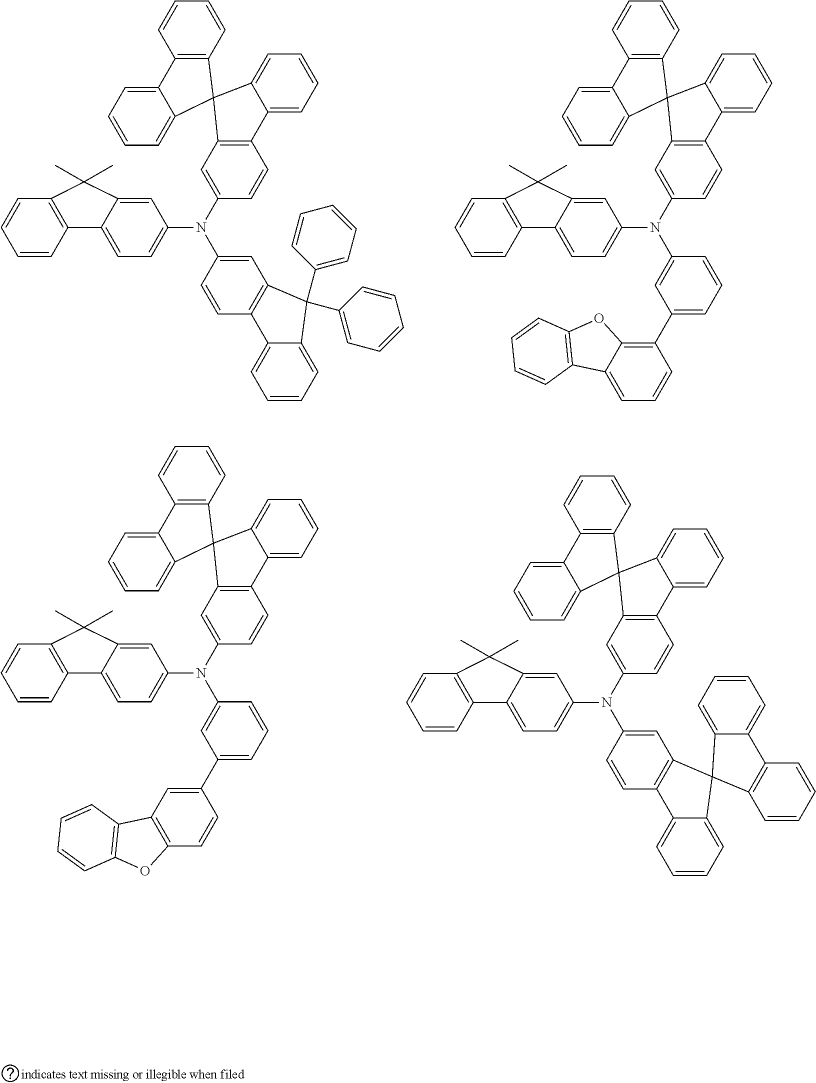 [SMILES: CC1(C)c2ccccc2-c2ccc(N(c3ccc4c(c3)C(c3ccccc3)(c3ccccc3)c3ccccc3-4)c3ccc4c(c3)C3(c5ccccc5-c5ccccc53)c3ccccc3-4)cc21 ; CC1(C)c2ccccc2-c2ccc(N(c3cccc(-c4cccc5c4oc4ccccc45)c3)c3ccc4c(c3)C3(c5ccccc5-c5ccccc53)c3ccccc3-4)cc21 ; CC1(C)c2ccccc2-c2ccc(N(c3cccc(-c4ccc5oc6ccccc6c5c4)c3)c3ccc4c(c3)C3(c5ccccc5-c5ccccc53)c3ccccc3-4)cc21 ; CC1(C)c2ccccc2-c2ccc(N(c3ccc4c(c3)C3(c5ccccc5-c5ccccc53)c3ccccc3-4)c3ccc4c(c3)C3(c5ccccc5-c5ccccc53)c3ccccc3-4)cc21]

[symbol]

In the invention, the hole-injecting layer contains a compound represented by the following formula (100). Preferably, the hole-injecting layer essentially consists of this compound.

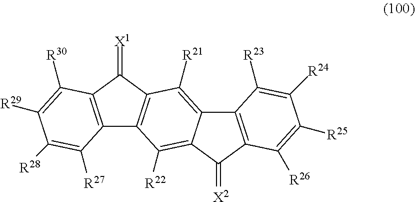

In the formula (100), X¹ and X² are independently represented by the following formula (a) or (b). X¹ and X² are preferably represented by the formula (a).

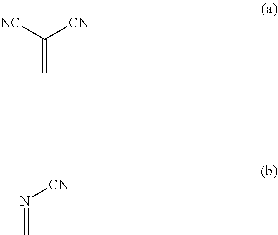

In the formula (100), R²¹ to R³⁰ are independently a hydrogen atom, a substituted or unsubstituted alkyl group including 1 to 20 carbon atoms, a substituted or unsubstituted cycloalkyl group including 3 to 20 carbon atoms, a substituted or unsubstituted alkenyl group including 1 to 20 carbon atoms, a substituted or unsubstituted aryl group including 6 to 50 ring carbon atoms, a substituted or unsubstituted heterocyclic group including 3 to 50 ring atoms, a halogen atom, a substituted or unsubstituted fluoroalkyl group including 1 to 20 carbon atoms, a substituted or unsubstituted alkoxy group including 1 to 20 carbon atoms, a substituted or unsubstituted fluoroalkoxy group including 1 to 20 carbon atoms, a substituted or unsubstituted aryloxy group including 6 to 50 ring carbon atoms, a substituted or unsubstituted aralkyloxy group including 7 to 70 carbon atoms, a substituted or unsubstituted amino group or a cyano group.

It is preferred that R²¹ to R³⁰ be independently a hydrogen atom, a fluorine atom, a fluoroalkyl group, a fluoroalkoxy group, a cyano group, or an aryl group that may be substituted by one or more substituents selected from a fluorine atom, a fluoroalkyl group, a fluoroalkoxy group and a cyano group.

Further, it is preferred that at least one of R²¹ to R³⁰ be a fluorine atom, a fluoroalkyl group, a fluoroalkoxy group, a cyano group, or an aryl group that may be substituted by one or more substituents selected from a fluorine atom, a fluoroalkyl group, a fluoroalkoxy group and a cyano group. By allowing them to be substituents, it is possible to enhance electron acceptability, to obtain an appropriate sublimation temperature or to suppress crystallization.

It is preferred that the compound containing the indenofluorenedione structure represented by the formula (100) be represented by the following formula (101).

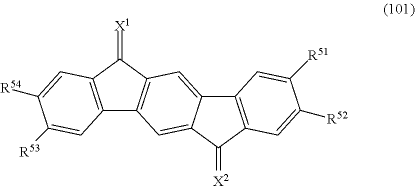

In the formula (101), R⁵¹ to R⁵⁴ are independently a hydrogen atom, a fluorine atom, a fluoroalkyl group, a fluoroalkoxy group, a cyano group, or an aryl group that may be substituted by one or more substituents selected from a fluorine atom, a fluoroalkyl group, a fluoroalkoxy group and a cyano group.

In the formula (101), it is preferred that one of R⁵¹ and R⁵² be an aryl group that may be substituted by one or more substituents selected from a fluorine atom, a fluoroalkyl group, a fluoroalkoxy group and a cyano group, and the other be a hydrogen atom, and that one of R⁵³ and R⁵⁴ be an aryl group that may be substituted by one or more substituents selected from a fluorine atom, a fluoroalkyl group, a fluoroalkoxy group and a cyano group, and the other be a hydrogen atom.

X¹ and X² have the same meaning as X¹ and X² in the formula (100). X¹ and X² are preferably represented by the formula (a).

Specific examples of the formula (100) are given below. However, the compound represented by the formula (100) is not limited thereto.

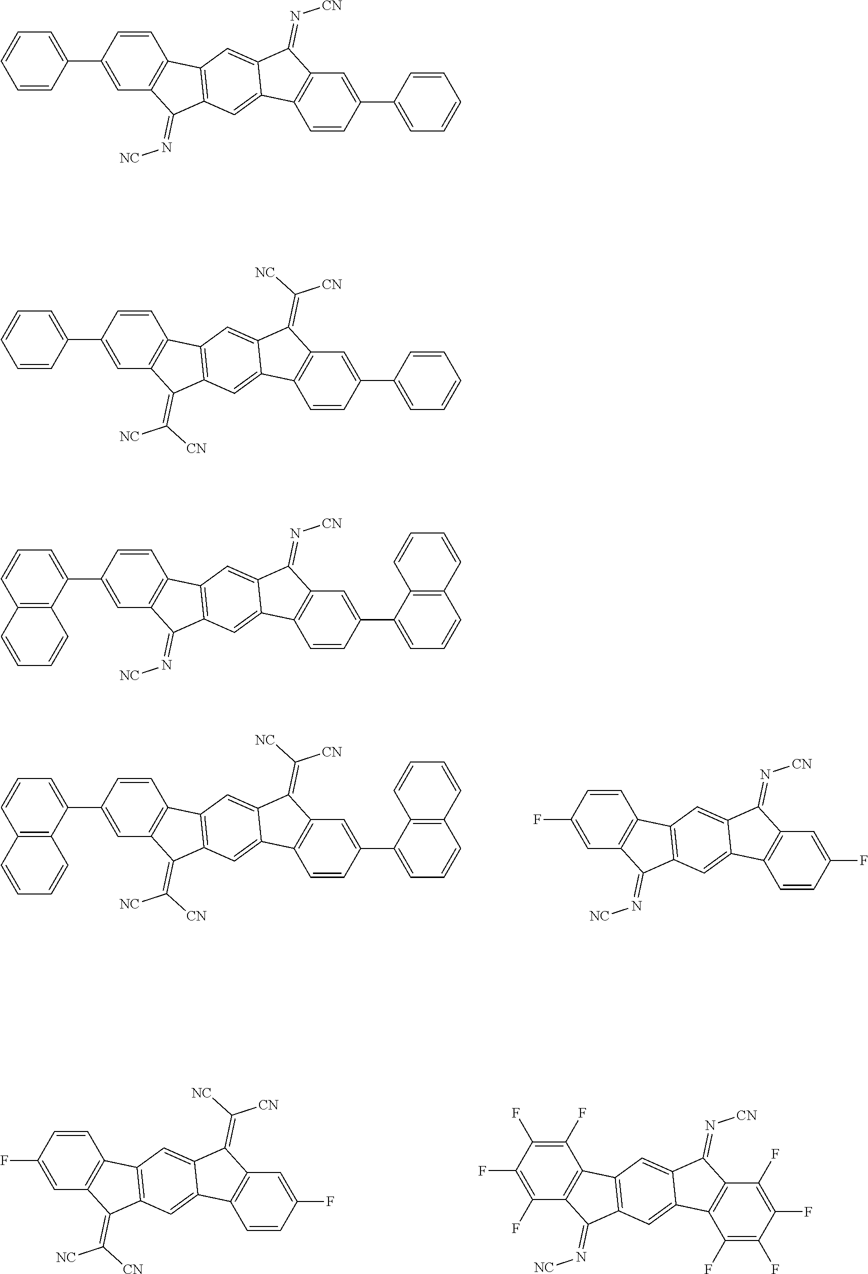

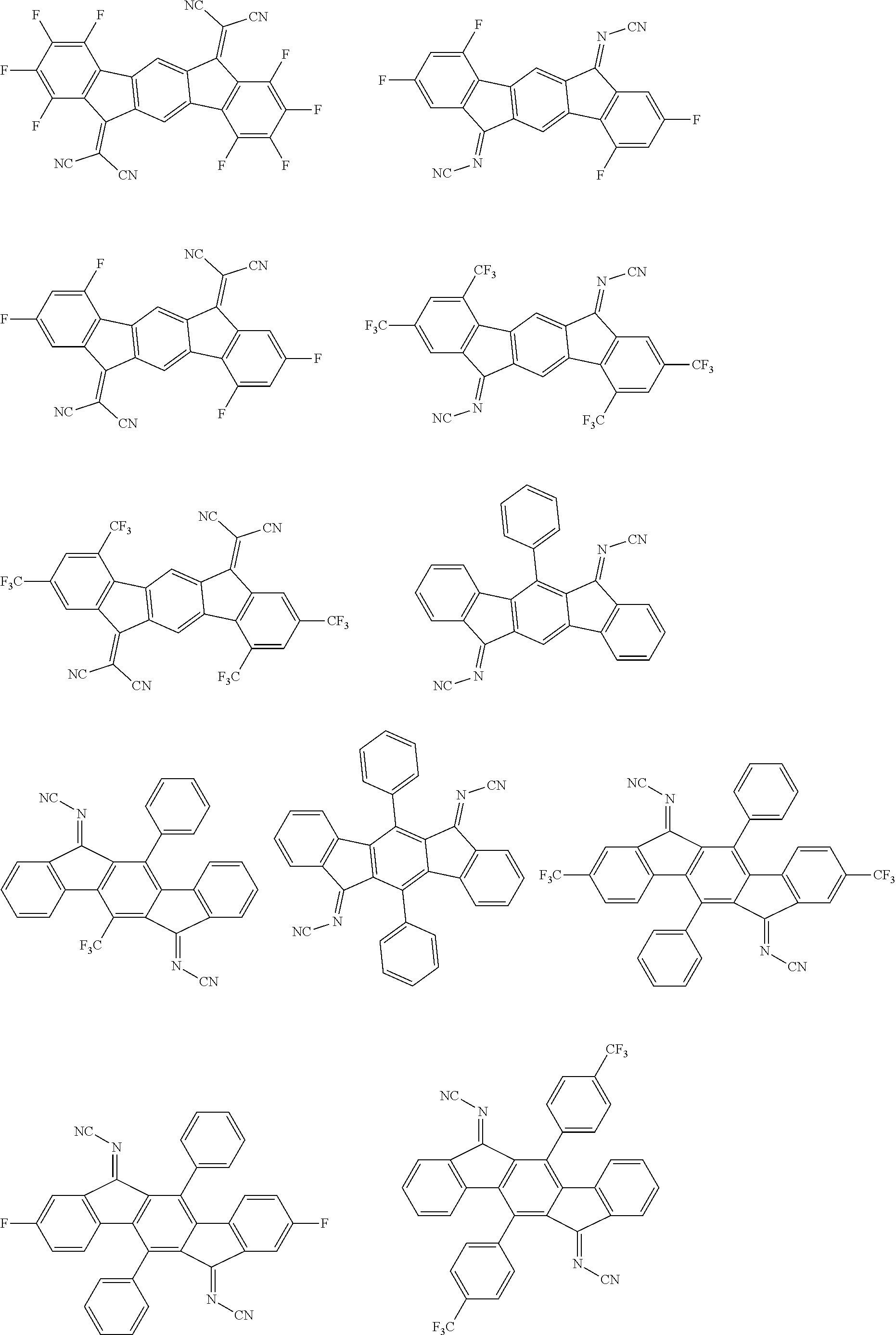

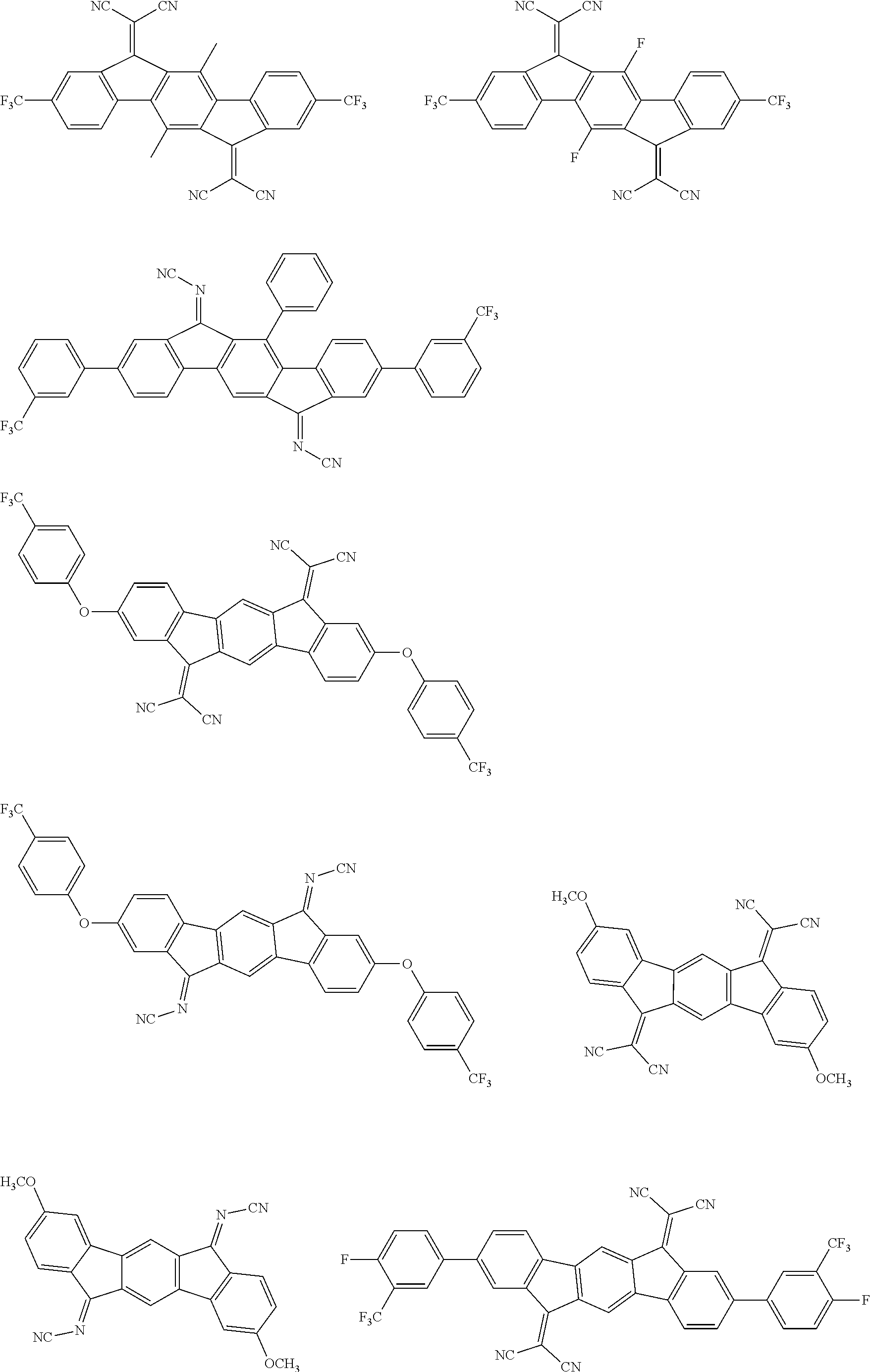

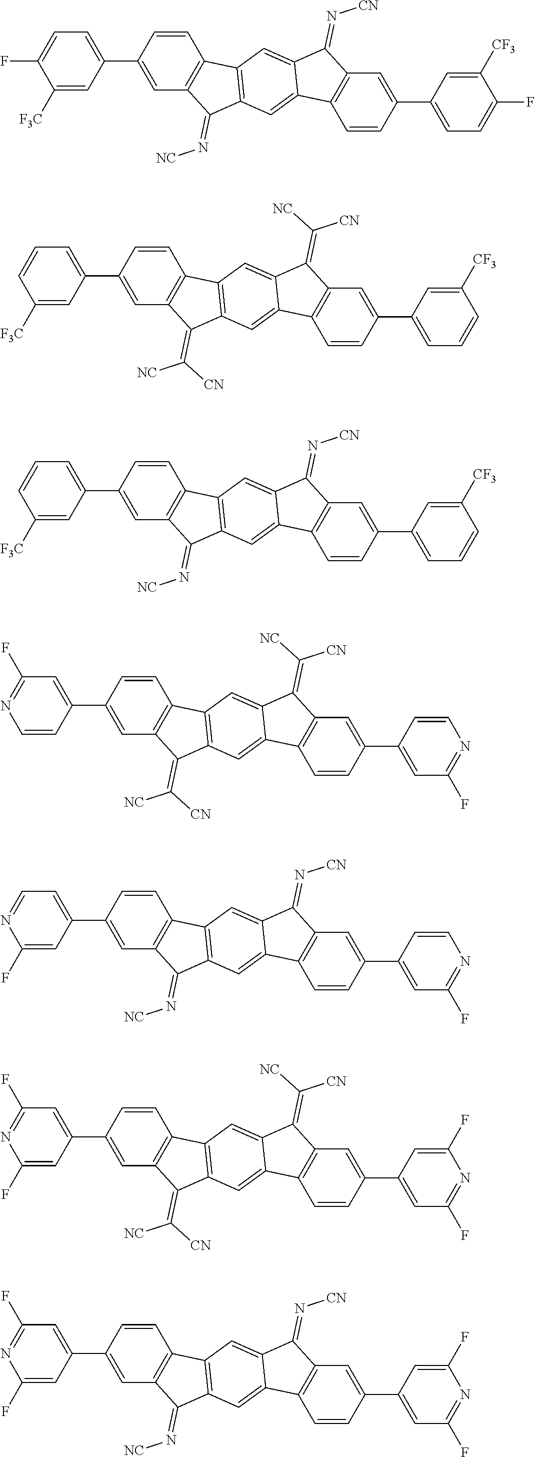

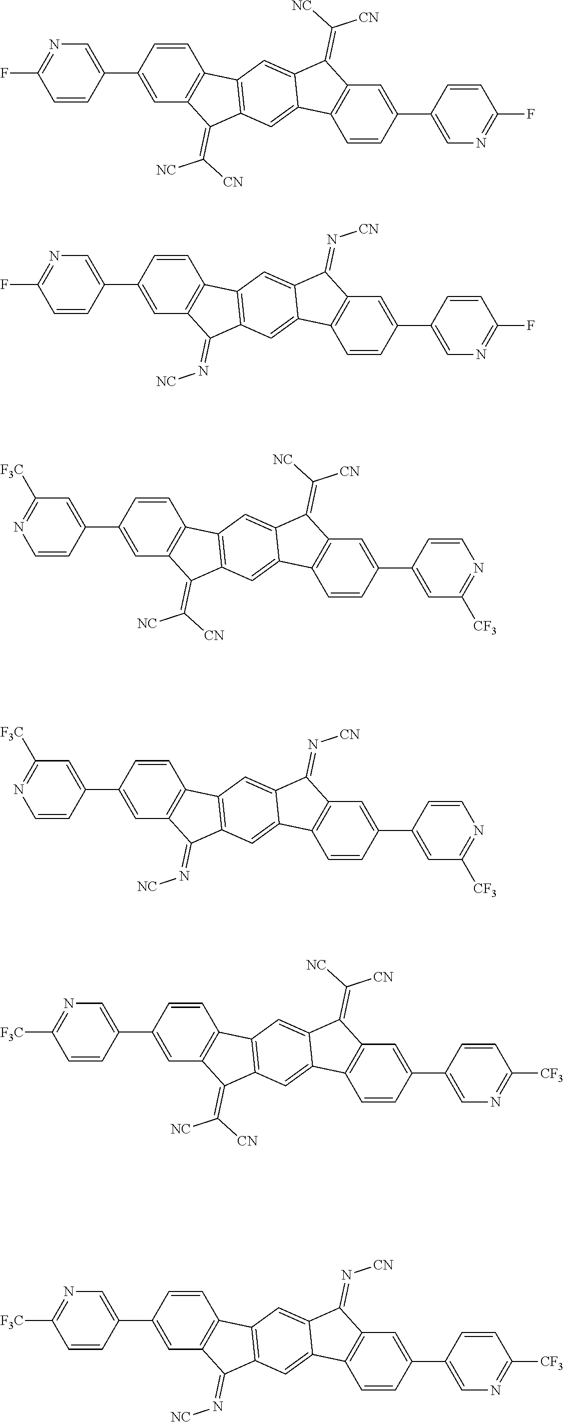

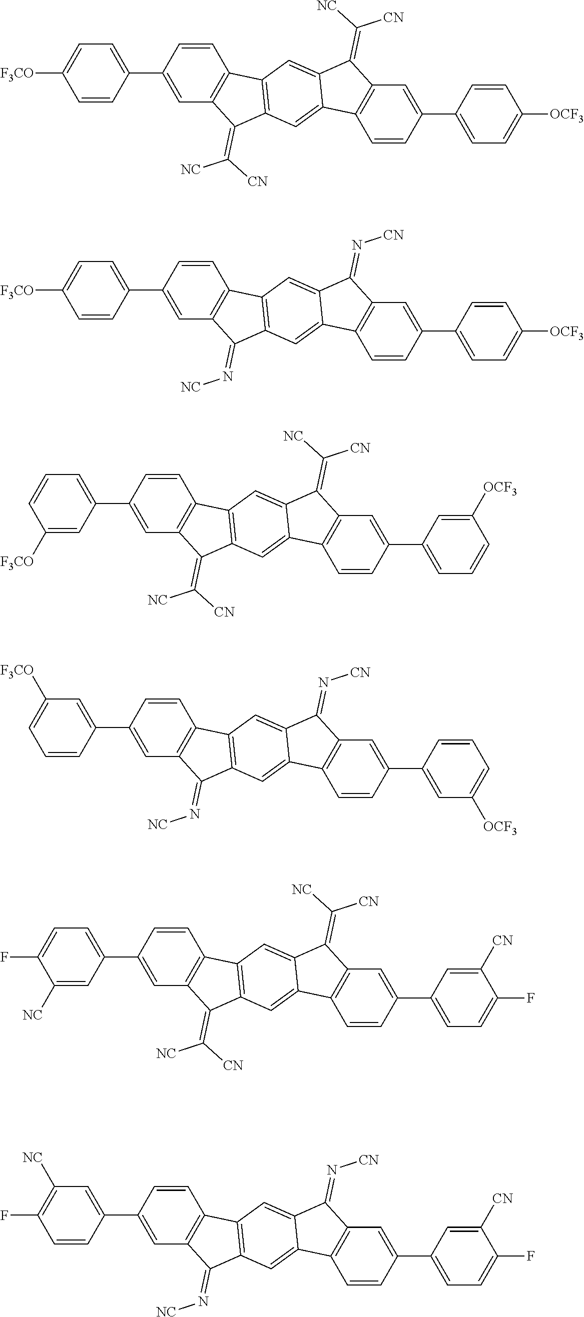

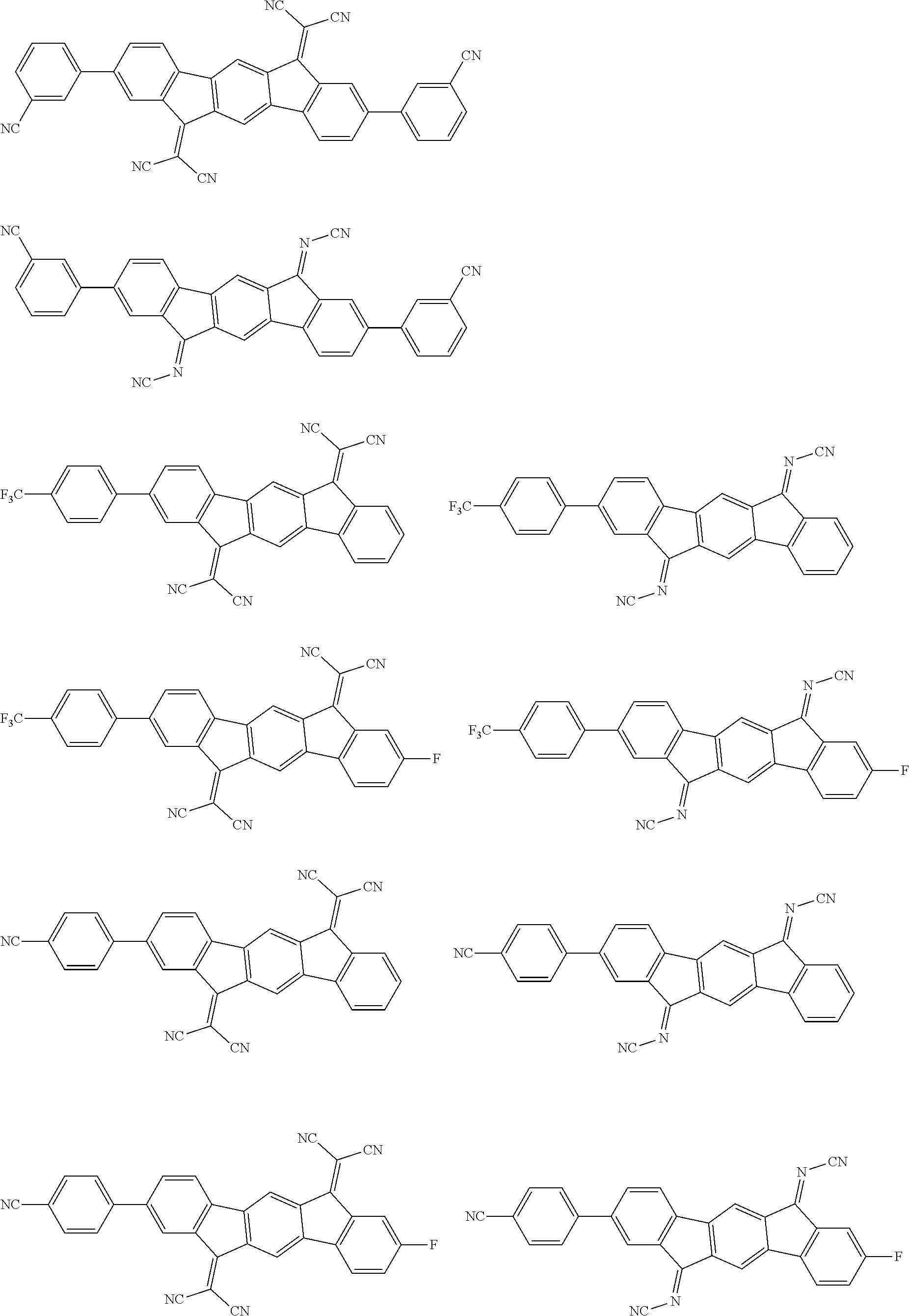

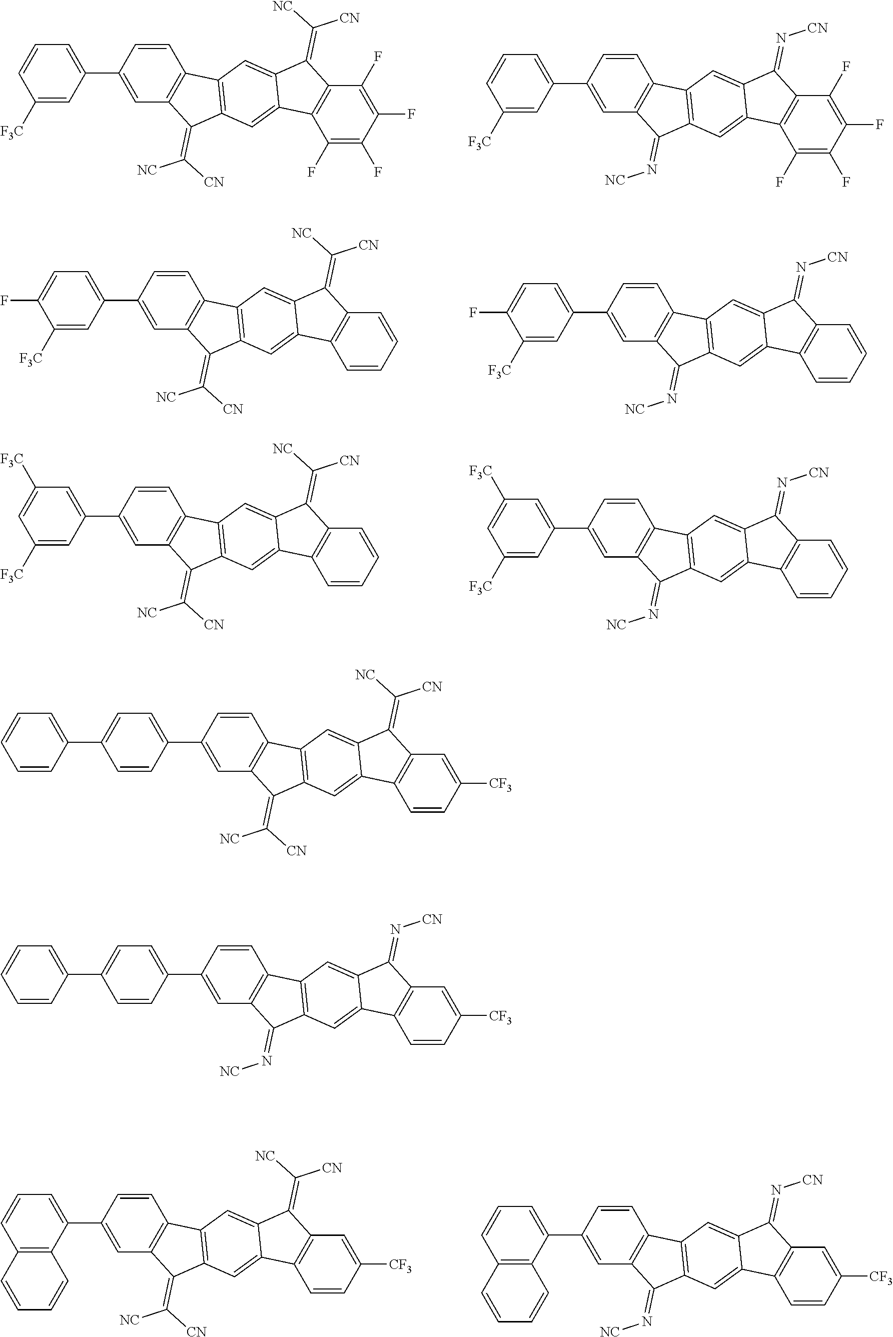

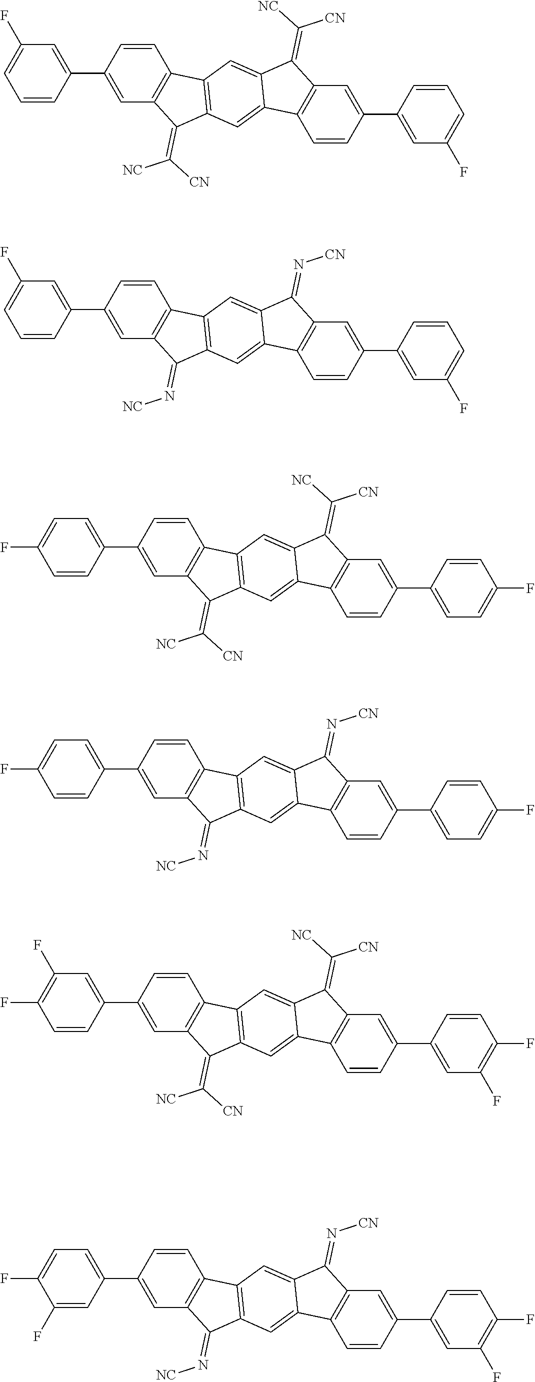 [SMILES: N#CC(C#N)=C1c2cc(-c3cccc(F)c3)ccc2-c2cc3c(cc21)-c1ccc(-c2cccc(F)c2)cc1C3=C(C#N)C#N ; N#C/N=c1\c2cc(-c3cccc(F)c3)ccc2c2cc3/c(=N/C#N)c4cc(-c5cccc(F)c5)ccc4c3cc12 ; N#CC(C#N)=C1c2cc(-c3ccc(F)cc3)ccc2-c2cc3c(cc21)-c1ccc(-c2ccc(F)cc2)cc1C3=C(C#N)C#N ; N#C/N=c1\c2cc(-c3ccc(F)cc3)ccc2c2cc3/c(=N/C#N)c4cc(-c5ccc(F)cc5)ccc4c3cc12 ; N#CC(C#N)=C1c2cc(-c3ccc(F)c(F)c3)ccc2-c2cc3c(cc21)-c1ccc(-c2ccc(F)c(F)c2)cc1C3=C(C#N)C#N ; N#C/N=c1\c2cc(-c3ccc(F)c(F)c3)ccc2c2cc3/c(=N/C#N)c4cc(-c5ccc(F)c(F)c5)ccc4c3cc12]

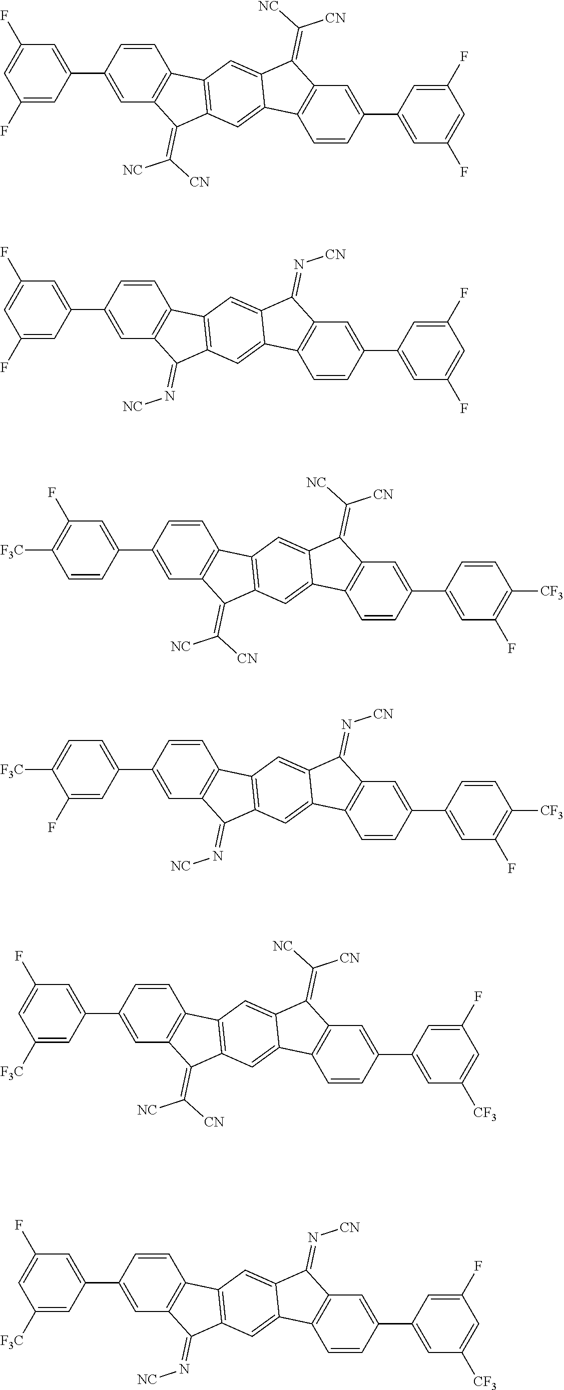

Preferable examples or specific examples of each group in the formula (1) excluding Ar¹, Ar² and L are shown below.

The alkyl portion of the alkyl group and the fluoroalkyl group, the alkyl portion of the alkoxy group, the alkyl portion of the fluoroalkoxy group and the alkyl portion of the alkoxycarbonyl group each include 1 to 20, preferably 1 to 8, and more preferably 1 to 4 carbon atoms, for example.

The cycloalkyl group includes 3 to 20, preferably 5 to 10, more preferably 5 to 6 carbon atoms, for example.

The alkenyl group includes 2 to 20, preferably 2 to 8, more preferably 2 to 4 carbon atoms, for example.

The aryl group or the arylene group includes 6 to 50, preferably 6 to 24, more preferably 6 to 12 ring carbon atoms, for example.

As for the aralkyloxy group, the alkyl portion includes 1 to 20, preferably 1 to 8, more preferably 1 to 4 carbon atoms, for example. The aryl portion includes 6 to 50, preferably 6 to 24, and more preferably 6 to 12 ring carbon atoms, for example.

The heterocyclic group, the heteroaryl group or the heteroarylene group includes 3 to 50, preferably 3 to 24, more preferably 3 to 12 ring atoms, for example. The heterocyclic group, the heteroaryl group or the heteroarylene group may include 5 to 50 ring atoms. In this case, the number of ring atoms is preferably 5 to 24, more preferably 5 to 12.

The aryloxy group includes 6 to 50, preferably 6 to 24, and more preferably 6 to 12 carbon atoms, for example.

As the alkyl group (alkyl portion), a methyl group, an ethyl group, a n-propyl group, an isopropyl group, a n-butyl group, an isobutyl group, a s-butyl group, a t-butyl group, a pentyl group (including isomers), a hexyl group (including isomers), a heptyl group (including isomers), an octyl group (including isomers), a nonyl group (including isomers), a decyl group (including isomers), an undecyl group (including isomers), and a dodecyl group (including isomers) or the like can be given, for example. A methyl group, an ethyl group, a n-propyl group, an isopropyl group, a n-butyl group, an isobutyl group, a s-butyl group, a t-butyl group and a pentyl group (including isomers) are preferable. A methyl group, an ethyl group, a n-propyl group, an isopropyl group, a n-butyl group, an isobutyl group, a s-butyl group and a t-butyl group are more preferable, with a methyl group and a t-butyl group being particularly preferable.

Examples of the (substituted) aryl group include a phenyl group, a naphthylphenyl group, a biphenylyl group, a terphenylyl group, a biphenylenyl group, a naphthyl group, a phenylnaphthyl group, an acenaphthylenyl group, an anthryl group, a benzoanthryl group, an aceanthryl group, a phenanthryl group, a benzophenanthryl group, a phenalenyl group, a fluorenyl group, a 9,9-dimethylfluorenyl group, a 7-phenyl-9,9-dimethylfluorenyl group, a pentacenyl group, a picenyl group, a pentaphenyl group, a pyrenyl group, a chrysenyl group, a benzochrysenyl group, an s-indacenyl group, an as-indacenyl group, a fluoranthenyl group, a perylenyl group, a fluorophenyl group, a trifluoromethylphenyl group, a (trifluoromethyl) fluorophenyl group, a trifluorophenyl group, a bis(trifluoromethyl)phenyl group, a (trifluoromethyl)difluorophenyl group, a trifluoromethoxyphenyl group, a trifluoromethoxyfluorophenyl group or the like. A phenyl group, a naphthylphenyl group, a biphenylyl group, a terphenylyl group, a naphthyl group, a 9,9-dimethylfluorenyl group are preferable. A phenyl group, a biphenylyl group, a naphthyl group and a 9,9-dimethylfluorenyl group are more preferable, with a phenyl group being particularly preferable. The same can be applied to the (substituted) arylene group.

The halogen atom is a fluorine atom, a chlorine atom, a bromine atom and an iodine atom, with a fluorine atom being particularly preferable.

As the fluoroalkyl group, a group obtained by substituting at least one, preferably 1 to 7, hydrogen atoms of the above-mentioned alkyl group including 1 to 20 carbon atoms by a fluorine atom can be given, for example. A heptafluoropropyl group, a pentafluoroethyl group, a 2,2,2-trifluoroethyl group, a trifluoromethyl group, a perfluorocyclohexyl group, a perfluoroadamantyl group are preferable. A pentafluoroethyl group, a 2,2,2-trifluoroethyl group and a trifluoromethyl group are more preferable, with a trifluoromethyl group being particularly preferable.

As the alkoxy group, a t-butoxy group, a propoxy group, an ethoxy group and a methoxy group are preferable. An ethoxy group and a methoxy group are more preferable, with a methoxy group being particularly preferable.

As the fluoroalkoxy group, a heptafluoropropoxy group, a pentafluoroethoxy group, a 2,2,2-trifluoroethoxy group, a trifluoromethoxy group, a 2,2,3,3,3-pentafluoropropoxy group, a 2,2,3,3-tetrafluoropropoxy group, a 1,1,1,3,3,3-hexafluoropropan-2-yloxy group are preferable. A pentafluoroethoxy group, a 2,2,2-trifluoroethoxy group and a trifluoromethoxy group are more preferable, with a trifluoromethoxy group being particularly preferable.

The heterocydic group contains at least one, preferably 1 to 3 hetero atoms, e.g. a nitrogen atom, a sulfur atom and an oxygen atom. As the heterocyclic group, a pyrrolyl group, a furyl group, a thienyl group, a thiophenyl group, a pyridyl group, a pyridazinyl group, a pyrimidinyl group, a pyrazinyl group, a triazinyl group, an imidazolyl group, an oxazolyl group, a thiazolyl group, a pyrazolyl group, an isoxazolyl group, an isothiazolyl group, an oxadiazolyl group, a thiadiazolyl group, a triazolyl group, an indolyl group, an isoindolyl group, a benzofuranyl group, an isobenzofuranyl group, a benzothiophenyl group, an indolizinyl group, a quinolizinyl group, a quinolyl group, an isoquinolyl group, a cinnolyl group, a phthalazinyl group, a quinazolinyl group, a quinoxalinyl group, a benzimidazolyl group, a benzoxazolyl group, a benzthiazolyl group, an indazolyl group, a benzisoxazolyl group, a benzisothiazolyl group, a carbazolyl group, a dibenzofuranyl group, a dibenzthiophenyl group, a phenanthridinyl group, an acridinyl group, a phenanthrolinyl group, a phenazinyl group, a phenothiazinyl group, a phenoxazinyl group, a xanthenyl group or the like can be given, for example. A furyl group, a thienyl group, a pyridyl group, a pyridazinyl group, a pyrimidinyl group, a pyrazinyl group, a triazinyl group, a benzofuranyl group, a benzothiophenyl group, a dibenzofuranyl group and a dibenzothiophenyl group are more preferable. The same can be applied to the (substituted) heteroarylene group.

As the aryloxy group, a terphenyloxy group, a biophenyloxy group and a phenoxy group are preferable. A biphenyloxy group and a phenoxy group are more preferable, with a phenoxy group being particularly preferable.

As the cycloalkyl group, a cyclopentyl group, a cyclohexy group or the like can be given.

As the alkenyl group, a vinyl group, a propenyl group (including a positional isomer of a double bond), a butenyl group (including a positional isomer of a double bond), a pentenyl group (including a positional isomer of a double bond) or the like can be given.

As examples of the (substituted) aralkyloxy group, a benzyloxy group, a pentafluorobenzyloxy group, a 4-trifluoromethylbenzyloxy group or the like can be given.

As examples of the (substituted) amino group, an amino group, a mono- or dimethylamino group, a mono- or diethylamino group, a mono- or diphenylamino group or the like can be given.

The arbitrary substituent in the “substituted or unsubstituted” mentioned above is selected from the group consisting of, an alkyl group including 1 to 50 (preferably 1 to 10, more preferably 1 to 5) carbon atoms; a fluoroalkyl group including 1 to 50 (preferably 1 to 10, more preferably 1 to 5) carbon atoms; a cydoalkyl group including 3 to 50 (preferably 3 to 6, more preferably 5 or 6) ring carbon atoms; an aryl group including 6 to 50 (preferably 6 to 24, more preferably 6 to 12) ring carbon atoms; an aralkyl group including 1 to 50 (preferably 1 to 10, more preferably 1 to 5) carbon atoms that has an aryl group including 6 to 50 (preferably 6 to 24, more preferably 6 to 12) ring carbon atoms; an amino group; a mono- or dialkylamino group having an alkyl group including 1 to 50 (preferably 1 to 10, more preferably 1 to 5) carbon atoms; a mono- or diarylamino group having an aryl group including 6 to 50 (preferably 6 to 24, more preferably 6 to 12) ring carbon atoms; an alkoxy group having an alkyl group including 1 to 50 (preferably 1 to 10, more preferably 1 to 5) carbon atoms; a fluoroalkoxy group having an alkyl group including 1 to 50 (preferably 1 to 10, more preferably 1 to 5) carbon atoms; an aryloxy group having an aryl group including 6 to 50 (preferably 6 to 24, more preferably 6 to 12) ring carbon atoms; a mono-, di- or tri-substituted silyl group having a group selected from an alkyl group including 1 to 50 (preferably 1 to 10, more preferably 1 to 5) carbon atoms and an aryl group including 6 to 50 (preferably 6 to 24, more preferably 6 to 12) ring carbon atoms; a heteroaryl group containing 1 to 5 (preferably 1 to 3, more preferably 1 to 2) hetero atoms (nitrogen atom, oxygen atom, sulfur atom) and including 5 to 50 (preferably 5 to 24, more preferably 5 to 12) ring atoms; a halogen atom (fluorine atom, chlorine atom, bromine atom, iodine atom); a cyano group; and a nitro group.

These substituents may be further substituted by the above-mentioned substituents. Further, a plurality of these substituents may be bonded with each other to form a ring.

In the hole-transporting zone, it is preferred that the hole-transporting layer containing the compound represented by the formula (1) and the hole-injecting layer containing the compound represented by the formula (100) be in contact with each other.

Hereinbelow, an explanation will be made on layers other than those in the hole-transporting zone that constitute an organic EL device. The layers and the materials that constitute the organic EL device are not restricted to those mentioned below

### (Substrate)

The substrate is used as a base of an emitting element. As the substrate, glass, quarts, plastic or the like can be used, for example. A flexible substrate may be used. A flexible substrate is a substrate that can be bent. For example, a plastic substrate made of polycarbonate or polyvinyl chloride or the like can be given.

### (Anode)

For an anode formed on the substrate, it is preferable to use a metal, an alloy, an electrically conductive compound having a large work function (specifically, 4.0 eV or more), a mixture thereof or the like. Specifically, indium oxide-tin oxide (ITO: Indium Tin Oxide), indium oxide-tin oxide containing silicon or silicon oxide, indium oxide-zinc oxide, tungsten oxide, indium oxide containing zinc oxide, graphene or the like can be given, for example. In addition, gold (Au), platinum (Pt) or a nitride of a metal material (e.g. titanium nitride) or the like can be given.

### (Light-Emitting Unit)

The organic EL device according to one aspect of the invention may be a fluorescent or phosphorescent monochromatic light-emitting device or a fluorescent/phosphorescent hybrid white light-emitting device. It may be of a simple type having a single light-emitting unit or may be of a tandem type having plural light-emitting units.

Here, the “light-emitting unit” means the minimum unit containing organic thin film layers that includes one or more layers, in which at least one of them is an emitting layer and light is emitted by re-combination of injected holes and injected electrons.

For example, as the representative device configuration of a simple type organic EL device, the following device configurations can be given.

### (1) Anode/Light-Emitting Unit/Cathode

The above-mentioned light-emitting unit may be of a stacked layer type structure in which plural phosphorescent emitting layers or plural fluorescent emitting layers are provided. In this case, a space layer may be provided between the emitting layers in order to prevent excitons generated in the phosphorescent emitting layer from scattering to the fluorescent emitting layer. The representative device configurations of the simple type light-emitting unit are shown below.

(a) (Hole-injecting layer/) Hole-transporting layer/Fluorescent emitting layer (/Electron-transporting layer)
(b) (Hole-injecting layer/) Hole-transporting layer/First phosphorescent fluorescent emitting layer/Second phosphorescent fluorescent emitting layer (/Electron-transporting layer)
(c) (Hole-injecting layer/) Hole-transporting layer/Phosphorescent emitting layer/Space layer/Fluorescent emitting layer (/Electron-transporting layer)
(d) (Hole-injecting layer/) Hole-transporting layer/First phosphorescent emitting layer/Second phosphorescent emitting layer/Space layer/Fluorescent emitting layer (/Electron-transporting layer)
(e) (Hole-injecting layer/) Hole-transporting layer/First phosphorescent emitting layer/Space layer/Second phosphorescent emitting layer/Space layer/Fluorescent emitting layer (/Electron-transporting layer)
(f) (Hole-injecting layer/) Hole-transporting layer/Phosphorescent emitting layer/Space layer/First fluorescent emitting layer/Second fluorescent emitting layer (/Electron-transporting layer)
(g) (Hole-injecting layer/) Hole-transporting layer/Electron-barrier layer/Fluorescent emitting layer (/Electron-transporting layer)
(h) (Hole-injecting layer/) Hole-transporting layer/Fluorescent emitting layer/Hole-barrier layer (/Electron-transporting layer)
(i) (Hole-injecting layer/) Hole-transporting layer/Fluorescent emitting layer/Triplet-barrier layer (/Electron-transporting layer)

The phosphorescent emitting layers or the fluorescent emitting layers mentioned above can emit different colors of light. Specifically, in the stacked layer light-emitting unit (d), layer configurations such as (Hole-injecting layer/) Hole-transporting layer/First phosphorescent emitting layer (red emission)/Second phosphorescent emitting layer (green emission)/Space layer/Fluorescent emitting layer (blue emission)/Electron-transporting layer, or the like can be given.

Between each emitting layer and the hole-transporting layer or the space layer, an electron-barrier layer may be appropriately provided. Further, between each emitting layer and the electron-transporting layer, a hole-barrier layer may be appropriately provided. By providing an electron-barrier layer or a hole-barrier layer, it is possible to confine electrons or holes in the emitting layer to enhance the re-combination probability of charges in the emitting layer, whereby the luminous efficiency can be improved.

As the representative device configuration of the tandem type organic EL device, the following device configurations can be given.

### (2) Anode/First Emitting Unit/Intermediate Layer/Second Emitting Unit/Cathode

### (3) Anode/First Emitting Unit/First Intermediate Layer/Second Emitting Unit/Second Intermediate Layer/Third Emitting Unit/Cathode

As the first emitting unit and the second emitting unit, they can independently be selected from the above-mentioned emitting units, for example.

The intermediate layer, the first intermediate layer and the second intermediate layer are generally called an intermediate electrode, an intermediate conductive layer, a charge-generation layer, an electron-withdrawing layer, a connection layer and an intermediate insulating layer. A known material configuration that the first intermediate layer supplies electrons to the first emitting unit and supplies holes to the second emitting unit, and the second intermediate layer supplies electrons to the second emitting unit and supplies holes to the third emitting unit can be used.

In the tandem type organic EL device, the hole-transporting layer containing the compound represented by the formula (1) and the hole-injecting layer containing the compound represented by the formula (100) may be included in at least one same emitting unit among two or more emitting units.

### (Guest Material of Emitting Layer)

The emitting layer is a layer that contains a material having a high emitting property, and various materials can be used. For example, as the material having a high emitting property, a fluorescent compound that emits fluorescent light or a phosphorescent compound that emits phosphorescent light can be used. A fluorescent compound is a compound that can emit light from the singlet excited state, and a phosphorescent compound is a compound that can emit light from the triplet excited state.

As the blue fluorescent emitting material that can be used in the emitting layer, a pyrene derivative, a styrylamine derivative, a chrysene derivative, a fluoranthene derivative, a fluorene derivative, a diamine derivative, a triarylamine derivative or the like can be used. As the green fluorescent emitting material that can be used in the emitting layer, an aromatic amine derivative or the like can be used. As the red fluorescent emitting material that can be used in the emitting layer, a tetracene derivative, a diamine derivative or the like can be given.

As the blue phosphorescent emitting material that can be used in the emitting layer, a metal complex such as an iridium complex, an osmium complex and a platinum complex is used. As the green phosphorescent emitting material that can be used in the emitting layer, an iridium complex or the like are used. As the red phosphorescent emitting material that can be used in the emitting layer, a metal complex such as an iridium complex, a platinum complex, a terbium complex and a europium complex can be given.

### (Host Material of Emitting Layer)

The emitting layer may have a configuration in which the above-mentioned material having a high emitting property (guest material) is dispersed in other materials (host material). As the material for dispersing the material having a high emitting property, various materials can be used. It is preferable to use a material having a higher lowest unoccupied molecular orbital level (LUMO level) and a lower highest occupied molecular orbital level (HOMO level) as compared with a material having a higher emitting property.

As the material (host material) for dispersing a material having a high emitting property, 1) metal complex such as an aluminum complex, a beryllium complex and a zinc complex, 2) a heterocyclic compound such as an oxadiazole derivative, a benzoimidazole derivative and a phenanthroline derivative, 3) a fused aromatic compound such as a carbazole derivative, an anthracene derivative, a phenanthrene derivative, a pyrene derivative and a chrysene derivative, and 3) an aromatic amine compound such as a triarylamine derivative and a fused polycyclic aromatic amine derivative are used.

### (Electron-Transporting Layer)

The electron-transporting layer is a layer that contains a material having high electron-transporting property. In the electron-transporting layer, 1) a metal complex such as an aluminum complex, a beryllium complex and a zinc complex, 2) a heterocydic aromatic compound such as an imidazole derivative, a benzimidazole derivative, an azine derivative, a carbazole derivative and a phenanthroline derivative and 3) a polymer compound can be used.

### (Electron-Injecting Layer)

An electron-injecting layer is a layer that contains a material having a high electron-injecting property. In the electron-injecting layer, an alkali metal or an alkaline earth metal, such as lithium (Li), lithium fluoride (LiF), cesium fluoride (CsF), calcium fluoride (CaF₂) and lithium oxide (LiOx), or a compound of these can be used.

### (Cathode)

For a cathode, it is preferable to use a metal having a small work function (specifically, 3.8 eV or less), an alloy, an electrically conductive compound, a mixture of those or the like. As specific examples of the cathode material, an element belonging to Group 1 or Group 2 of the periodic table of the elements, i.e., an alkali metal such as lithium (Li) or cesium (Cs), an alkaline earth metal such as magnesium (Mg), alloys containing those (e.g. MgAg, AlLi), and a rare earth metal and an alloy containing those or the like can be given.

The organic EL device of the invention is used in various display apparatuses or various light-emitting apparatuses. For example, it can be used as a light source such as a planar luminous body or a backlight for display apparatus, a display part of a portable phone, PDA, car navigation, an instrumental panel of a vehicle, lighting or the like.

### EXAMPLES

The compounds used in the Examples and the Comparative Examples are as follows:

### [Hole-Injecting Compound]

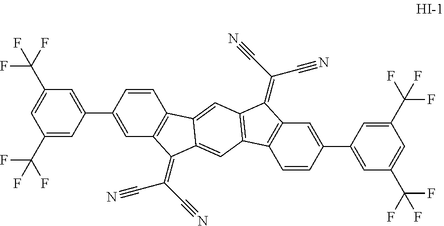 [SMILES: N#CC(C#N)=C1c2cc(-c3cc(C(F)(F)F)cc(C(F)(F)F)c3)ccc2-c2cc3c(cc21)-c1ccc(-c2cc(C(F)(F)F)cc(C(F)(F)F)c2)cc1C3=C(C#N)C#N]

### [Hole-Transporting Compound]

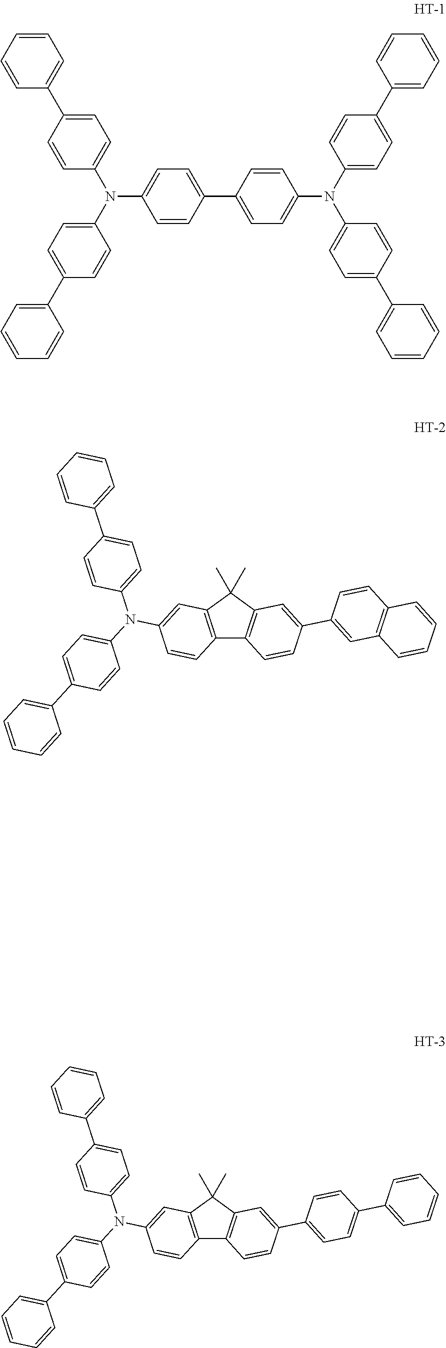 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; CC1(C)c2cc(-c3ccc4ccccc4c3)ccc2-c2ccc(N(c3ccc(-c4ccccc4)cc3)c3ccc(-c4ccccc4)cc3)cc21 ; CC1(C)c2cc(-c3ccc(-c4ccccc4)cc3)ccc2-c2ccc(N(c3ccc(-c4ccccc4)cc3)c3ccc(-c4ccccc4)cc3)cc21]

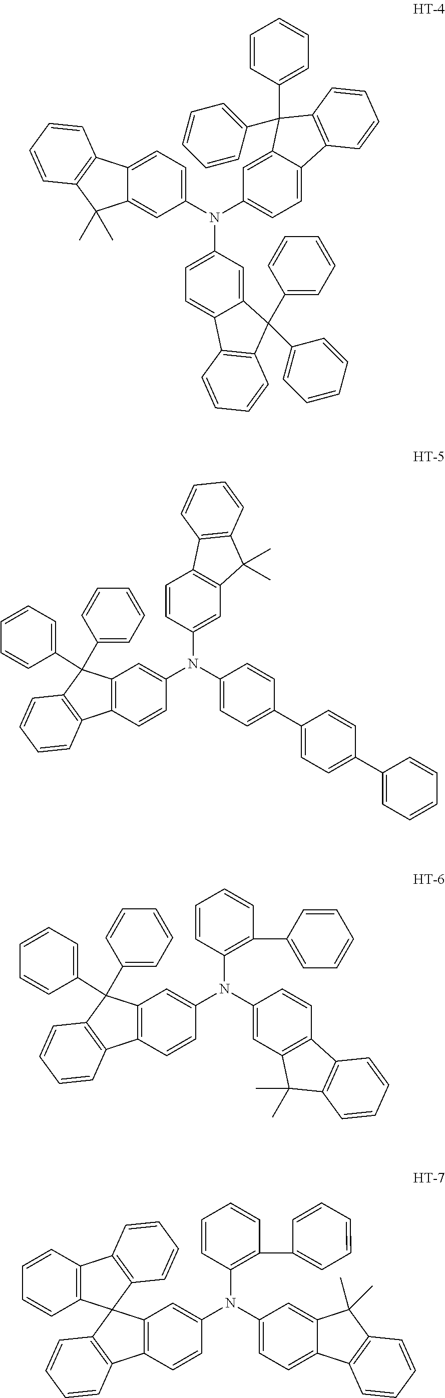 [SMILES: CC1(C)c2ccccc2-c2ccc(N(c3ccc4c(c3)C(c3ccccc3)(c3ccccc3)c3ccccc3-4)c3ccc4c(c3)C(c3ccccc3)(c3ccccc3)c3ccccc3-4)cc21 ; CC1(C)c2ccccc2-c2ccc(N(c3ccc(-c4ccc(-c5ccccc5)cc4)cc3)c3ccc4c(c3)C(c3ccccc3)(c3ccccc3)c3ccccc3-4)cc21 ; CC1(C)c2ccccc2-c2ccc(N(c3ccc4c(c3)C(c3ccccc3)(c3ccccc3)c3ccccc3-4)c3ccccc3-c3ccccc3)cc21 ; CC1(C)c2ccccc2-c2ccc(N(c3ccc4c(c3)C3(c5ccccc5-c5ccccc53)c3ccccc3-4)c3ccccc3-c3ccccc3)cc21]

### [Host Compound and Dopant Compound]

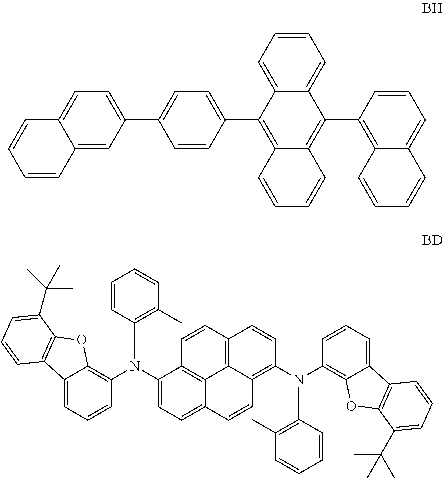

### [Electron-Transporting Compound]

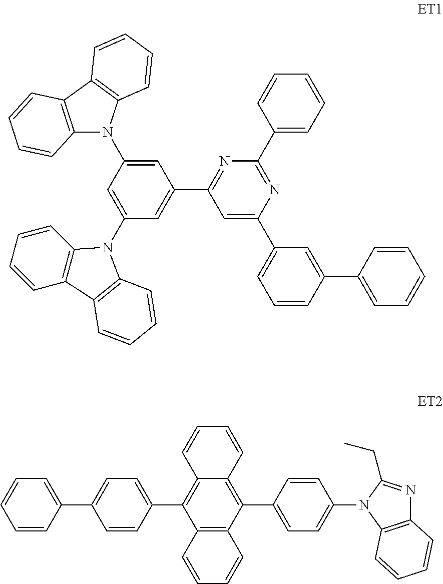 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; CCc1nc2ccccc2n1-c1ccc(-c2c3ccccc3c(-c3ccc(-c4ccccc4)cc3)c3ccccc23)cc1]

### Comparative Example 1

On an ITO substrate on which ITO (anode) was formed into a film, a hole-injecting compound HI-1, a hole-transporting compound HT-1, a host material BH and a dopant material BD (dopant material: 4 wt %), an electron-transporting compound ET-1, an electron-transporting compound ET-2, an electron-injecting compound LiF and aluminum as a cathode material were sequentially deposited and stacked, whereby a device having the following configuration was obtained. The number in the parenthesis indicates the thickness (unit: nm). ITO/HI-1(5)/HT-1(100)/BH:BD(25)(4%)/ET-1(10)/ET-2(15)/LiF(1)/Al(50)

### Examples 1 to 6

Devices were produced in the same manner as in Comparative Example 1, except that, in Comparative Example 1, the compounds shown in Table 1 were used as the hole-transporting compound.

For the obtained devices, the following evaluations were conducted. The results are shown in Table 1.

A voltage was applied to the resulting organic EL device such that the current density became 10 mA/cm², and the voltage at the time was measured. An EL emission spectrum at the time was also measured by means of a spectroradiometer (CS-1000 manufactured by Konica Minolta, Inc.). From the resulting spectral radiance, an external quantum efficiency EQE (%) was calculated.

The voltages and the external quantum efficiencies of the devices of Examples 1 to 2 when the voltage and the external quantum efficiency of the device of Comparative Example 1 were independently taken as 1.00 are shown in Table 1.

TABLE 1

| | Hole-transporting compound | Voltage | EQE |
|---|---|---|---|
| Comp. Ex. 1 | HT-1 | 1.00 | 1.00 |
| Ex. 1 | HT-2 | 0.96 | 1.15 |
| Ex. 2 | HT-3 | 0.98 | 1.20 |
| Ex. 3 | HT-4 | 0.98 | 1.30 |
| Ex. 4 | HT-5 | 0.99 | 1.20 |
| Ex. 5 | HT-6 | 0.99 | 1.20 |
| Ex. 6 | HT-7 | 0.99 | 1.20 |

Although only some exemplary embodiments and/or examples of this invention have been described in detail above, those skilled in the art will readily appreciate that many modifications are possible in the exemplary embodiments and/or examples without materially departing from the novel teachings and advantages of this invention. Accordingly, all such modifications are intended to be included within the scope of this invention.

The specification of the Japanese patent applications claiming the priority under the Paris Convention to the invention is incorporated herein by reference in its entirety.

1. An organic electroluminescence device comprising an anode, a hole-injecting layer, a hole-transporting layer, an emitting layer and a cathode in this order,
wherein
the hole-transporting layer comprises a compound represented by the following formula (1), and
the hole-injecting layer comprises a compound represented by the following formula (100): 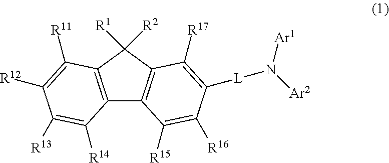
wherein in the formula (1), R¹ and R² are independently an alkyl group including 1 to 4 carbon atoms;
R¹¹ to R¹⁷ are independently a hydrogen atom or a substituent, and adjacent ones of R¹¹ to R¹⁴ may be bonded with each other to form a ring;
Ar¹ and Ar¹ are independently a substituted or unsubstituted aryl group including 6 to 25 ring carbon atoms or a substituted or unsubstituted heteroaryl group including 6 to 24 ring atoms; and
L is a single bond, a substituted or unsubstituted arylene group or a substituted or unsubstituted heteroarylene group; 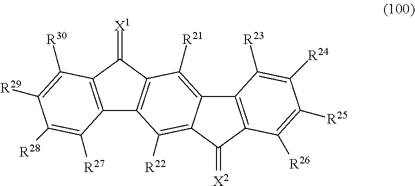
wherein in the formula (100), X¹ and X² are independently represented by the following formula (a) or (b); and
R²¹ to R³⁰ are independently a hydrogen atom, a substituted or unsubstituted alkyl group including 1 to 20 carbon atoms, a substituted or unsubstituted cycloalkyl group including 3 to 20 carbon atoms, a substituted or unsubstituted alkenyl group including 1 to 20 carbon atoms, a substituted or unsubstituted aryl group including 6 to 50 ring carbon atoms, a substituted or unsubstituted heterocyclic group including 3 to 50 ring atoms, a halogen atom, a substituted or unsubstituted fluoroalkyl group including 1 to 20 carbon atoms, a substituted or unsubstituted alkoxy group including 1 to 20 carbon atoms, a substituted or unsubstituted fluoroalkoxy group including 1 to 20 carbon atoms, a substituted or unsubstituted aryloxy group including 6 to 50 ring carbon atoms, a substituted or unsubstituted aralkyloxy group including 7 to 70 carbon atoms, a substituted or unsubstituted amino group or a cyano group.

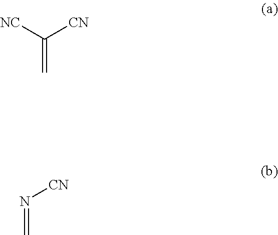

2. The organic electroluminescence device according to claim 1, wherein in the formula (1), Ar¹ and Ar² are independently a group selected from the following formulas (11) to (21): 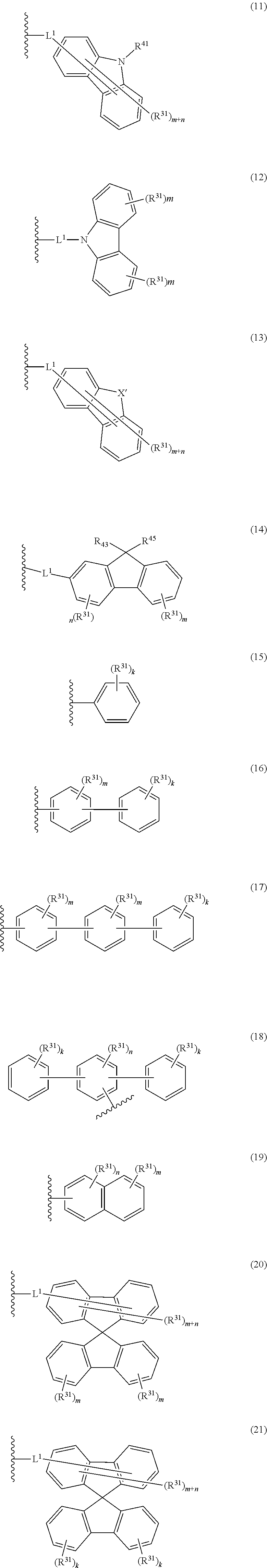
wherein in the formulas (11) to (21),
R³¹ is a substituted or unsubstituted alkyl group including 1 to 20 carbon atoms, a substituted or unsubstituted aryl group including 6 to 50 ring carbon atoms, a halogen atom, a substituted or unsubstituted fluoroalkyl group including 1 to 20 carbon atoms, a substituted or unsubstituted alkoxy group including 1 to 20 carbon atoms, a substituted or unsubstituted fluoroalkoxy group including 1 to 20 carbon atoms or a cyano group, and if plural R³¹s are present, the plural R³¹s may be the same or different;
k is an integer of 0 to 5, m is an integer of 0 to 4 and n is an integer of 0 to 3;
X′ is an oxygen atom or a sulfur atom;
L¹ is the same as L in the formula (1);
R⁴¹ is a hydrogen atom, a substituted or unsubstituted alkyl group including 1 to 20 carbon atoms, a substituted or unsubstituted aryl group including 6 to 50 ring carbon atoms, a halogen atom, a substituted or unsubstituted fluoroalkyl group including 1 to 20 carbon atoms, a substituted or unsubstituted alkoxy group including 1 to 20 carbon atoms, a substituted or unsubstituted fluoroalkoxy group including 1 to 20 carbon atoms or a cyano group;
R⁴³ and R⁴⁵ are independently a substituted or unsubstituted alkyl group including 1 to 20 carbon atoms, a substituted or unsubstituted heterocyclic group including 3 to 50 ring atoms, a halogen atom, a substituted or unsubstituted fluoroalkyl group including 1 to 20 carbon atoms, a substituted or unsubstituted alkoxy group including 1 to 20 carbon atoms, a substituted or unsubstituted fluoroalkoxy group including 1 to 20 carbon atoms, a substituted or unsubstituted aryloxy group including 6 to 50 ring carbon atoms or a cyano group, and R⁴³ and R⁴⁵ may form a ring;
wherein in the formula (11), R³¹ may be bonded to an arbitrary position of the carbazole skeleton, and L¹ may be bonded to an arbitrary position of the carbazole skeleton;
wherein in the formula (13), R³¹ may be bonded to an arbitrary position of the dibenzofuran skeleton or the dibenzothiophene skeleton, and L¹ may be bonded to an arbitrary position of the dibenzofuran skeleton or the dibenzothiophene skeleton;
wherein in the formula (20), R³¹ may be bonded to an arbitrary position of the fluorene skeleton, and L¹ may be bonded to an arbitrary position of the fluorene skeleton;
wherein in the formula (21), R³¹ may be bonded to an arbitrary position of the fluorene skeleton, and L¹ may be bonded to an arbitrary position of the fluorene skeleton; and
wherein in the formulas (12), (14) to (21), R³¹ may be bonded to an arbitrary position of the benzene ring, and L¹ may be bonded to an arbitrary position of the benzene ring.

3. The organic electroluminescence device according to claim 2, wherein Ar¹ is represented by the formula (16).

4. The organic electroluminescence device according to claim 2, wherein Ar¹ is represented by any one of the formulas (14) to (19).

5. The organic electroluminescence device according to claim 2, wherein Ar¹ and Ar² are independently represented by any one of the formulas (14) to (19).

6. The organic electroluminescence device according to claim 2, wherein Ar¹ and Ar² are independently represented by any one of the formulas (11) to (13).

7. The organic electroluminescence device according to claim 2, wherein Ar¹ and Ar² are independently represented by any one of the formulas (20) and (21).

8. The organic electroluminescence device according to claim 2, wherein Ar¹ is represented by the following formula in which L¹ is bonded at the 3rd position of the carbazole skeleton:

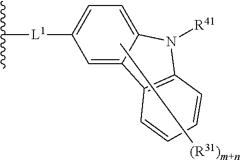

9. The organic electroluminescence device according to claim 3, wherein Ar¹ and Ar² are the same.

10. The organic electroluminescence device according to claim 2, wherein Ar¹ is represented by the formula (16) and Ar² is represented by any one of the formulas (14) to (21).

11. The organic electroluminescence device according to claim 2, wherein Ar¹ is represented by any one of the formulas (14) to (19) and Ar² is represented by any one of the formulas (11) to (13).

12. The organic electroluminescence device according to claim 1, wherein in the formula (1), L is a group represented by the following formula (22): 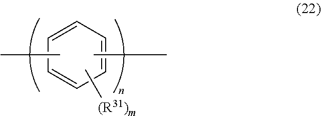
wherein in the formula, R³¹is a substituted or unsubstituted alkyl group including 1 to 20 carbon atoms, a substituted or unsubstituted aryl group including 6 to 50 ring carbon atoms, a halogen atom, a substituted or unsubstituted fluoroalkyl group including 1 to 20 carbon atoms, a substituted or unsubstituted alkoxy group including 1 to 20 carbon atoms, a substituted or unsubstituted fluoroalkoxy group including 1 to 20 carbon atoms or a cyano group, and when plural R³¹s are present, the plural R³¹s may be the same or different;
m is an integer of 0 to 4, and n is an integer of 0 to 3; and
when n is 0, L is a single bond; and
when n is plural, the plural R³¹s may be the same or different and the two or more ms may be the same or different.

13. The organic electroluminescence device according to claim 12, wherein, in the formula (22), when n is plural, the plural benzene rings are bonded with each other at the para-position.

14. The organic electroluminescence device according to claim 1, wherein in the formula (1), L is a single bond.

15. The organic electroluminescence device according to claim 1, wherein in the formula (1), the substituent of the “substituted or unsubstituted” of Ar¹, Ar² and L and the substituent of R¹¹ to R¹⁷ are independently one or more selected from a substituted or unsubstituted aryl group including 6 to 50 ring carbon atoms, a substituted or unsubstituted alkyl group including 1 to 20 carbon atoms, a substituted or unsubstituted alkoxy group including 1 to 20 carbon atoms, a substituted or unsubstituted aryloxy group including 6 to 50 ring carbon atoms, a substituted or unsubstituted arylthio group including 6 to 50 ring atoms, a substituted or unsubstituted alkoxycarbonyl group including 2 to 50 carbon atoms, an amino group that is substituted by a substituted or unsubstituted aryl group including 6 to 50 ring carbon atoms, a halogen atom, a cyano group, a nitro group, a hydroxyl group and a carboxyl group.

16. The organic electroluminescence device according to claim 1, wherein in the formula (100), R²¹ to R³⁰ are independently a hydrogen atom, a fluorine atom, a fluoroalkyl group, a fluoroalkoxy group, a cyano group, or
an aryl group that may be substituted by one or more substituents selected from a fluorine atom, a fluoroalkyl group, a fluoroalkoxy group and a cyano group.

17. The organic electroluminescence device according to claim 1, wherein the compound represented by the formula (100) is a compound represented by the following formula (101): 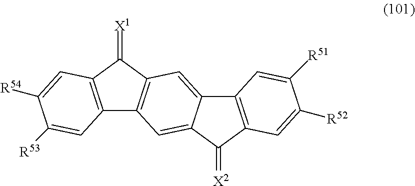
wherein in the formula, R⁵¹ to R⁵⁴ are independently a hydrogen atom, a fluorine atom, a fluoroalkyl group, a fluoroalkoxy group, a cyano group, or
an aryl group that may be substituted by one or more substituents selected from a fluorine atom, a fluoroalkyl group, a fluoroalkoxy group and a cyano group; and
X¹ and X² are the same as X¹ and X² in the formula (100).

18. The organic electroluminescence device according to claim 17, wherein in the formula (101), one of R⁵¹ and R⁵² is an aryl group that may be substituted by one or more substituents selected from a fluorine atom, a fluoroalkyl group, a fluoroalkoxy group and a cyano group, and the other is a hydrogen atom, and
one of R⁵³ and R⁵⁴ is an aryl group that may be substituted by one or more substituents selected from a fluorine atom, a fluoroalkyl group, a fluoroalkoxy group and a cyano group, and the other is a hydrogen atom.

19. The organic electroluminescence device according to claim 1, wherein X¹ and X² in the formula (100) or (101) is represented by the formula (a).

20. The organic electroluminescence device according to claim 1, wherein the emitting layer comprises one or more compounds selected from a pyrene derivative, a styrylamine derivative, a chrysene derivative, a fluoranthene derivative, a fluorene derivative, a diamine derivative and a triarylamine derivative.

21. The organic electroluminescence device according to claim 1, wherein the emitting layer comprises one or more compounds selected from an iridium complex, an osmium complex and a platinum complex.

22. The organic electroluminescence device according to claim 1, wherein the organic electroluminescence device comprises two or more of the hole-transporting layers, and
any one of the two or more hole-transporting layers comprises the compound represented by the formula (1).

23. A display apparatus or a light-emitting apparatus that is provided with the organic electroluminescence device according to claim 1.
